(19) United States
(12) Patent Application Publication
(10) Pub. No.: US 2008/0056993 A1
(43) Pub. Date: Mar. 6, 2008

(54) Compositions, Methods and Kits Using Adenosine and Inosine in Combination for Diagnosis and Treatment

(75) Inventor: Philippe Gorny, Paris (FR)

Correspondence Address: DECHERT LLP, P.O. BOX 390460, MOUNTAIN VIEW, CA, 94039-0460, US

(73) Assignee: ADENOBIO N.V., Amsterdam Schiphol (NL)

(21) Appl. No.: 11/693,967

(22) Filed: Mar. 30, 2007

Related U.S. Application Data
(60) Provisional application No. 60/787,771, filed on Mar. 31, 2006.

(51) Int. Cl. A61K 31/70; A61K 49/00; A61P 9/00

(52) U.S. Cl. 424/9.2; 514/45; 514/46

(57) ABSTRACT

The present disclosure relates to compositions, methods, and kits using adenosine and inosine in combination for diagnosis and treatment.

### 1. CROSS REFERENCE TO RELATED APPLICATIONS

This application claims benefit under 35 U.S.C. §119(e) to application Ser. No. 60/787,771, filed Mar. 31, 2006, the contents of which are incorporated herein by reference.

### 2. BACKGROUND

The purine nucleoside, adenosine, is widely used in clinical practice, both for diagnosis of cardiac and coronary abnormalities in pharmacological stress test protocols, and to treat supraventricular tachycardia. In addition to its strong vasodilatory action and its negative chronotropic effects, studies have shown that adenosine has protective effects against reperfusion injury following ischemic insult in the heart, brain and spinal cord, has powerful anti-inflammatory activity, stimulates repair processes, inhibits platelet aggregation and is capable of reducing pain and anesthesia requirements during surgery.

However, notwithstanding its extremely short half life (on the order of several seconds), side effects hinder the use of adenosine at doses that provide maximal efficacy. For example, when administered by continuous intravenous infusion at the dose recommended for pharmacologic stress testing, 140 μg/kg/min for 6 minutes, adenosine causes frequent side effects. See, Adenoscan® (adenosine injection) product label. Side-effects, including subjective symptoms such as sensation of heat, flushing, dyspnea and chest pain, are dose-related: at a dosage of 70 μg/kg/min or less, adenosine adverse reactions are very few and of mild intensity. However, when given for stress testing by intravenous perfusion at 70 μg/kg/min or less, or even at 90-120 μg/kg/min, adenosine shows reduced efficacy, and is not recommended for clinical use at such reduced dosages.

In an effort to reduce side effects at maximally effective agonist doses, adenosinergic agents are being developed that are selective for various of the adenosine receptor subtypes. See, e.g., U.S. Pat. Nos. 7,019,027; 6,531,457; 6,448,235; 6,322,771; and 5,877,180. However, these compounds have a much longer half life than adenosine, and tend to induce the side effects associated with the receptor subtype for which they are specific—e.g., flushing, headache, and dyspnea upon stimulation of the adenosine A₂ₐ receptor, or chest pain after stimulation of the adenosine A₁ receptor. Thus, although more specific, these agents are more likely to trigger prolonged side effects, and to require administration of pharmacologic antidotes, than is adenosine itself, whose side effects rapidly dissipate once administration is stopped. Moreover, none of these selective agents has yet been approved for clinical use. There thus exists a continuing need in the art for injectable adenosinergic agonists that can be used with maximal efficacy, and that can managed clinically in the same manner as adenosine, yet with reduced side effects.

### 3. SUMMARY

As set forth in the Examples below, it has now been discovered that inosine sufficiently potentiates and/or modulates certain adenosine actions at selected adenosine:inosine weight ratios as to permit adenosine to be used at reduced dosage with reduced side effects, yet maximal efficacy. The combination of these two natural nucleosides, each with well-established pharmacokinetic and safety profiles, finds immediate use in diagnosis and treatment.

Accordingly, described herein are compositions, kits, and methods useful for exploiting the vasodilating effects, the cell protective activities, and the cell repair abilities of adenosine and inosine at dosages in which adenosine's most frequent side effects are significantly reduced or avoided, while maintaining maximum efficacy in terms of the treatment and diagnosis of injured tissues.

In one aspect, pharmaceutical compositions are provided comprising both adenosine and inosine (the combination of adenosine and inosine in a single composition hereinafter, “BIDOSINE”).

In one series of embodiments, the adenosine:inosine (A:I) weight ratio is between 1:1 to 1:20, with typical embodiments having A:I ratios of from 1:1 to 1:6. In another series of embodiments, the A:I ratio is between about 1:1 to about 20:1, with typical embodiments having A:I ratios of 4:1 to 7:1.

In various embodiments, the pharmaceutical compositions are suitable for intravenous, intra-atrial, or intra-arterial infusion.

In typical embodiments, adenosine and inosine are present at concentrations suitable for intravenous administration at an adenosine dosage rate of 10-100 μg/kg/min and inosine dosage rate of 10-2000 μg/kg/min. In some embodiments, the pharmaceutical composition comprises adenosine and inosine at concentrations suitable for intravenous administration at an adenosine dosage rate of 35-70 μg/kg/min and inosine dosage rate of 35-350 μg/kg/min. In yet other embodiments, the pharmaceutical composition comprises adenosine and inosine at concentrations suitable for intravenous administration at an adenosine dosage rate of 10-30 μg/kg/min and an inosine dosage rate of 200-600 μg/kg/min.

In various embodiments, the pharmaceutical composition comprises adenosine at a concentration of about 0.5 to 4 mg/ml. In some embodiments, the pharmaceutical composition comprises inosine at a concentration of about 0.3 to about 20 mg/ml.

In another aspect, unit doses of the pharmaceutical composition are provided containing 7-30 ml of the pharmaceutical composition as a sterile, nonpyrogenic, fluid suitable for parenteral administration. In some embodiments, the unit dose contains about 5 ml, 10 ml, or 15 ml. In other embodiments, the unit dose contains about 200-750 ml.

In another aspect, methods are provided in which adenosine and inosine are concurrently infused, either in a single “BIDOSINE” pharmaceutical composition, or in separate compositions, by a parenteral route. In some embodiments, for example, adenosine is infused at 35-70 μg/kg/min, with inosine infused at an A:I ratio of 2:1 to 10:1 (e.g., in one series of embodiments, adenosine at 50-70 μg/kg/min, with inosine at 10-20 μg/kg/min).

The pharmaceutical compositions described herein can be administered by intravenous, intra-atrial or intra-arterial continuous infusion to a mammal. In some embodiments, the pharmaceutical compositions are administered to an awake mammal. In other embodiments, the pharmaceutical compositions are administered to an anesthetized mammal undergoing surgery.

In some embodiments, the pharmaceutical compositions are administered to humans. Typical dosages for intravenous administration to humans comprise adenosine at the dosage of 10 to 100 μg/kg/min with inosine at the dosage of 10 to 600 μg/kg/min. In various embodiments, dosages for administration to humans comprise adenosine at 35-70 μg/kg/min and inosine at 35-350 μg/kg/min. In some other embodiments adenosine is infused at 10-30 μg/kg/min and inosine dosages at 200-600 μg/kg/min. In some particular embodiments, adenosine is infused at 40-70 μg/kg/min with inosine at 10-20 μg/kg/min.

Typical dosages for selective intra-arterial administration to humans (one minute or more) comprise adenosine at the dosage of 20 to 40 μg/min with inosine at the dosage of 20 to 100 μg/min. In various embodiments, dosages for selective intra-arterial infusion, comprise adenosine at 30 μg/min and inosine at 30 to 60 μg/min.

In one aspect, an improved method is provided for pharmacologic stress testing, the improvement comprising concurrently administering adenosine and inosine to induce the pharmacologic stress, wherein adenosine and inosine are administered in an adenosine:inosine ratio of about 1:1 to about 1:20.

In some embodiments, a pharmaceutical composition comprising adenosine and inosine is administered as the stressor, and the presence and/or severity of myocardial dysfunction assessed using single photon emission computed tomography (SPECT) or positron emission tomography (PET).

In other aspect, concurrent parenteral administration of adenosine and inosine, e.g., via the pharmaceutical compositions described herein, are used to treat acute inflammatory and reperfusion diseases, including but not limited to, acute coronary syndromes with or without myocardial infarction, stroke, limb ischemia, spinal cord injury or ischemia, acute pancreatitis, mesenteric ischemia.

In one series of embodiments, for example, methods are provided for treating post-ischemic myocardial injury. The methods comprise administering at least a first concurrent parenteral infusion of adenosine and inosine during or following an acute cardiac ischemic event. The adenosine:inosine ratio is about 1:1 to about 1:20, or conversely from about 1:1 to 20:1. In some embodiments, the acute ischemic event is a myocardial infarction.

In another aspect, methods are provided for treating acute injury to the central or peripheral nervous system. The methods comprise administering at least a first concurrent parenteral infusion of adenosine and inosine during or following an acute injury to the central or peripheral nervous system. In various embodiments, adenosine and inosine are infused at an A:I ratio of about 1:1 to about 1:20, or of about 1:1 to about 20:1.

In other aspects, concurrent parenteral administration of adenosine and inosine, e.g., via the pharmaceutical compositions described herein, are used to treat acute pulmonary resistance and/or to increase left ventricular ejection fraction and/or to increase cardiac output.

In other aspects, concurrent parenteral administration of adenosine and inosine—for example, by parenteral administration of the BIDOSINE compositions described herein—are administered to an anesthetized patient undergoing surgery to reduce pain.

In certain embodiments, the pharmaceutical compositions are administered to an anesthetized patient undergoing cardiac or neurologic surgical procedures to reduce the rate of postoperative complications such as myocardial infarction or cognitive damages.

In some embodiments the pharmaceutical compositions are administered to an anesthetized patient undergoing transplantation surgical procedures.

In various embodiments, adenosine and inosine are concurrently administered directly into the cerebrospinal fluid either intrathecally or using appropriate delivery methods for introduction into the cerebral ventricle or the cistema magna. In some embodiments, adenosine and inosine are administered in a single composition. For example, in some embodiments, the compositions are administered directly into the cerebral fluid to ensure a local concentration of adenosine and inosine in the 1 μm to 1 mM range.

### 4. BRIEF DESCRIPTION OF THE DRAWINGS

FIG. 1 illustrates that the maximal Carotid Blood Flow (CaBF) achievable in the rat by continuous administration of adenosine is obtained by infusion of adenosine at 0.1 mg/kg/min given for a 5 minute period. (The dose required to give maximal effect when adenosine is used as a single agent in an experimental or clinical setting is hereinafter referred to, with respect to that setting, as “adenosine max” or “adenosine max dose”). Data show the effects of increasing doses (each administered as a 5 min continuous i.v. infusion) of adenosine (0.01, 0.03, 0.1, 0.3 and 1 mg/kg/min) on CaBF (mL/min) in anaesthetized rat. Values are expressed as percentage variations from baseline (Mean±SEM, n=6).

FIGS. 2A-2D show the potentiating effect on CaBF (mL/min) resulting from combining a fixed dose of adenosine (0.05 mg/kg/min administered by continuous infusion, equal to half adenosine max) with inosine at different adenosine:inosine (A:I) weight ratios, as compared to CaBF at adenosine max.

FIG. 3 shows the synergistic effect on increase in CaBF of combining adenosine at half adenosine max with inosine at an A:I weight ratio of 1:4. Data show the effects on CaBF (mL/min) of 6 minute continuous i.v. infusions of adenosine (0.05 mg/kg/min), inosine (0.2 mg/kg/min), and adenosine (0.05 mg/kg/min)+inosine (0.2 mg/kg/min), on CaBF (mL/min) in anaesthetized rats. Values are expressed as percentage variations from baseline (Mean±SEM, n=6). The data demonstrate that combination of adenosine at half adenosine max (0.05 mg/kg/min) with inosine at 0.2 mg/kg/min (A:I weight ratio of 1:4) increased CaBF by 13±6%, as compared to an increase of 7±4% with adenosine alone at adenosine half max (0.05 mg/kg/min), and as compared to an increase of 4±2% with inosine alone at 0.2 mg/kg/min. Expressed as percentage increase from baseline, the increase in CaBF effected by the combination was greater than the sum of increases caused by adenosine and inosine as single agents.

FIG. 4 shows that the potentiating effect of combining adenosine with inosine is still seen when adenosine doses are reduced below half adenosine max. The data show the effects on CaBF (mL/min) of 6 minute continuous i.v. infusions of adenosine (0.1 mg/kg/min, adenosine max), adenosine (0.03 mg/kg/min), and adenosine (0.03 mg/kg/min)+inosine (0.25 mg/kg/min) in anaesthetized rats. In this example, potentiation is seen at an adenosine dose of 0.03 mg/kg/min, combined with inosine at 0.25 mg/kg/min, an A:I weight ratio of 1:8. Even at this reduced adenosine posology, the effect on CaBF is clearly potentiated by inosine. Values are expressed as percentage change from baseline (Mean±SEM, n=6).

FIG. 5 shows that inosine alone is poorly effective at increasing CaBF even at doses 10 times those at which adenosine alone is effective. Data show the effects on CaBF (mL/min) of increasing doses of inosine (0.3, 1, 3, 10 and 30 mg/kg/min), each administered as a 5 minute i.v. infusion, in anaesthetized rats. Values are expressed as absolute variations from baseline, in mL/min (Mean±SEM, n=6). No response is seen at 0.3 mg/kg/min. Modest decreases in CaBF are seen at 1 mg/kg/min (10 times adenosine max). Between 1 to 10 mg/kg/min, there is a sharp slump and dose-dependent decrease in CaBF.

FIGS. 6A-6F depict the effects of the combination of adenosine and inosine on mean arterial blood pressure and carotid vascular resistance. With adenosine fixed at half adenosine max, as A:I ratios increase from 1:1 to 1:3, the effect of the combination on (decreasing) arterial resistance and (decreasing) blood pressure progressively increases, equaling the effects of adenosine max on mean at an A:I ratio of 1:3. Panels 6A and 6B show effects, respectively, on mean arterial blood pressure and carotid vascular resistance of continuous infusions of adenosine alone (0.1 mg/kg/min, adenosine max), adenosine alone (0.05 mg/kg/min, half adenosine max), and adenosine at half adenosine max+inosine (0.05 mg/kg/min, for an A:I ratio of 1:1). Panels 6C and 6D respectively show effects on mean arterial pressure and carotid vascular resistance of continuous infusions of adenosine alone (0.1 mg/kg/min, adenosine max), adenosine alone (0.05 mg/kg/min, half adenosine max), and adenosine at half adenosine max+inosine (0.1 mg/kg/min, for an A:I ratio of 1:2). Panels 6E and 6F show effects on mean arterial pressure and carotid vascular resistance of continuous infusions of adenosine alone (0.1 mg/kg/min, adenosine max), adenosine alone (0.05 mg/kg/min, half adenosine max), and adenosine at half adenosine max+inosine (0.15 mg/kg/min, for an A:I ratio of 1:3).

FIGS. 7A-7D illustrate that, with adenosine at half adenosine max, at A:I ratios of 1:4 and above, effects on (decreasing) blood pressure and (decreasing) arterial resistance exceeds that of adenosine maximal.

FIGS. 8A and 8B show the effects of adenosine and BIDOSINE on left ventricular relaxation constant (Tau) in the sheep. Tau (left ventricular relaxation constant) is a parameter for relaxation. Tau is less load-dependent than dP/dtₘᵢₙ. The data demonstrate that adenosine at adenosine max and BIDOSINE (adenosine at half adenosine max, A:I ratio of 1:5) both increase ejection fraction and stimulate systolic function, and also improve diastolic function. The data further show that BIDOSINE performs slightly better than adenosine. Specifically, Tau is significantly shorter after adenosine and BIDOSINE compared with baseline (ANOVA p=0.001). The change in Tau by adenosine and BIDOSINE is comparable. This means that both products improve relaxation. This result appears contrary to the effect described by dP/dtₘᵢₙ (see Table 2), the discrepancy caused by the load-dependency of dP/dtₘᵢₙ, with BIDOSINE having less effect on loading conditions than adenosine.

FIGS. 9A-9C show that BIDOSINE 1:5 (adenosine at 0.05 mg/kg/min, half adenosine max, with inosine at 0.25 mg/kg/min), hastens the behavioral recovery in a rat model of spinal cord ischemia, with results equivalent on multiple tests to adenosine effects at 0.1 mg/kg/min. Gross and fine motor functional outcomes are compared between the different groups (n=12/group) after the injury until day 10 (BIDOSINE vs. saline, two way ANOVA p<0.001). Tests: panel 9A (open field test); panel 9B (inclined plane test); panel 9C (grid navigation).

FIGS. 10A-10B show that BIDOSINE 1:5 (adenosine at 0.05 mg/kg/min, half adenosine max, with inosine at 0.25 mg/kg/min), hastens the behavioral recovery in a rat model of spinal cord ischemia, with results equivalent on multiple tests to adenosine effects at 0.1 mg/kg/min. Panels 10A and 10B show proprioception comparison between the different groups.

FIG. 11 shows that BIDOSINE 1:5 (adenosine at 0.05 mg/kg/min, half adenosine max, with inosine at 0.25 mg/kg/min), hastens the behavioral recovery in a rat model of spinal cord ischemia, with results equivalent on multiple tests to adenosine effects at 0.1 mg/kg/min. Data compare bladder function (BIDOSINE vs. saline, two ways ANOVA p<0.001).

### 5. DETAILED DESCRIPTION

5.1 Overview

As set forth in the Examples below, it has now been discovered that the purine nucleoside, inosine, sufficiently potentiates the activity of adenosine at certain weight (or molar) ratios to permit adenosine to be used at reduced dosage, with reduced side effects, yet with maximal efficacy in diagnostic and therapeutic vasodilation, in cardiovascular therapy, for neuroprotection, and in treating various other acute pathological disorders.

At some ratios of adenosine to inosine (A:I), additive effects are seen. At other A:I ratios, synergistic effects are seen.

Throughout this disclosure, A:I ratios are expressed as ratios by weight. For example, in the context of relative concentrations in a single combined composition, or in the context of relative concentrations in separately administered compositions, A:I ratios expressed herein intend adenosine (mg):inosine (mg), or adenosine (mg/ml):inosine (mg/ml). In the context of intravenous infusion dosages, A:I ratios expressed herein intend adenosine (μg nucleoside/kg body weight/min):inosine (μg nucleoside/kg body weight/min). In the context of intra-arterial infusion dosages, such as intracoronary infusion dosages, A:I ratios expressed herein intend adenosine (μg/min):inosine (μg/min). Given the closeness in their molecular weights, 267.42 for adenosine, 268.27 for inosine, weight ratios closely approximate molar ratios.

In embodiments in which the clinical objective is coronary vasodilation or decrease of arterial resistance, optimal effects are typically observed at A:I ratios between 1:3 to 1:6. In embodiments in which the clinical goal is to effect cell protection, maintain cell function, and/or induce cell repair, optimal A:I ratios typically range from either 1:1 to 10:1, or conversely, from 1:15 to 1:20, with other ratios currently less favored.

Whatever the clinical goal, concurrent administration of inosine permits adenosine to be administered less than the dose required to provide either maximal vasodilation or optimal cell protection or cell repair when adenosine is used as a single agent (hereinafter, “adenosine max” or “adenosine max dose”), while retaining maximal efficacy.

For example, in human patients undergoing cardiac testing, the adenosine max for intravenous administration is approximately 140 μg/kg/min for six minutes; in certain embodiments, concurrent administration of inosine at preferred ratios permits adenosine to be administered efficaciously at 70 μg/kg/min for 4 minutes, or even less.

For example, in acute coronary syndrome, studies have shown that intravenous administration of adenosine as a single agent at 70 μg/kg/min for 3 hours is more effective than a three hour intravenous infusion at 50 μg/kg/min. Doses higher than 70 μg/kg/min were not tested, for fear of side effects. In certain embodiments, concurrent administration of inosine at preferred ratios permits adenosine to be administered efficaciously at 50 μg/kg/min for 3 hours or less.

Because inosine is extremely well tolerated, and adenosine side effects are dose-dependent, in various embodiments the methods and compositions of the present invention reduce the deleterious side effects observed in current practice.

Adenosine and inosine can be concurrently administered for the treatment or prevention of human acute inflammatory and reperfusion disease, of any etiology, for detecting the presence and/or assessing the severity of myocardial dysfunction, for decreasing pulmonary artery resistance and/or increasing left ventricular ejection fraction and/or increasing cardiac output in selected diseased patients, for reducing the rate of various specific complications during or after surgery, and for improving the delivery of stem cells to organs.

As used herein, “acute inflammatory disease” refers to any recently injured tissue or organ, whatever the cause (ischemia, infection, intoxication, trauma, inflammation, etc.), in which acute inflammatory processes and reduction of local blood flow take place, optionally with release of cytotoxic substances. Reperfusion disease refers to the restoration of blood flow to ischemic tissues which often results in events that extend injury beyond that observed with ischemia alone. “Acute inflammatory and reperfusion disease” includes conditions such as myocardial infarction, brain ischemia or spinal cord ischemia during surgical procedures, stroke, critical limb ischemia, spinal cord injury, acute pancreatitis, kidney ischemia, mesenteric ischemia or any reperfused organ during transplantation procedures.

5.2 Pharmaceutical Compositions

In a first aspect, the present invention provides pharmaceutical compositions comprising adenosine and inosine. Such compositions, irrespective of the absolute or relative amounts of adenosine and inosine, are referred to as BIDOSINE in this disclosure.

In typical embodiments, the pharmaceutical composition comprises adenosine and inosine in an adenosine:inosine (A:I) weight ratio of about 1:1 to about 1:20. In certain embodiments, the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, even 1:20, with nonintegral ratios between 1:1 and 1:20 permissible. In certain presently preferred embodiments, the composition comprises adenosine and inosine at a ratio of about 1:1 to 1:10, preferably about 1:3 to about 1:6. For certain clinical methods further described below, the composition usefully comprises adenosine and inosine at an A:I weight ratio of about 1:4 or 1:5.

In certain embodiments, the pharmaceutical compositions comprise adenosine and inosine at A:I weight ratios of about 20:1 to about 1:1. In certain embodiments, the ratio is about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, even 1:1, with nonintegral ratios between 20:1 and 1:1 permissible. In presently preferred embodiments, the A:I ratios are from about 2:1 to 10:1.

In certain embodiments, the pharmaceutical composition is suitable for intravenous, intra-atrial, or intra-arterial infusion.

The composition may, for example, be in the form of a sterile, nonpyrogenic, fluid composition.

In typical fluid embodiments, the concentration of adenosine is at least about 0.5 mg/ml, often at least about 1 mg/ml, 2 mg/ml, 3 mg/ml, even 4 mg/ml, 5 mg/ml, 6 mg/ml, or higher, with intermediate, nonintegral, values permissible. In certain embodiments, adenosine is present at a concentration of about 3 mg/ml.

In various embodiments, the concentration of inosine is at least about 0.3 mg/ml, and may usefully be as high as 20 mg/ml. The concentration may, in certain embodiments, be at least about 0.3 mg/ml, 0.4 mg/ml, 1 mg/ml, 2 mg/ml, 3 mg/ml, 4 mg/ml, 5 mg/ml, 6 mg/ml, or more, including 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, or 20 mg/ml, with intermediate, nonintegral values permissible.

In certain embodiments, the composition comprises adenosine at a concentration of about 3 mg/ml, and inosine at a concentration of about 9-18 mg/ml. In one embodiment, for example, the composition comprises adenosine at a concentration of about 3 mg/ml and inosine at a concentration of about 15 mg/ml. In another embodiment, the composition comprises adenosine at a concentration of about 3 mg/ml and inosine at a concentration of about 12 mg/ml.

In other embodiments, the composition is dry, and suitable for reconstitution prior to infusion by addition of a sterile fluid, such as saline. Usefully, the composition comprises adenosine and inosine in amounts suitable to permit reconstitution in the enclosing vessel to the adenosine and inosine concentrations above-described.

Whether fluid or dry, the composition may further comprise carriers and excipients suitable for intravenous, intra-atrial, or intra-arterial administration, as are well known in the art. See, Remington: The Science and Practice of Pharmacy, 21^{st} ed. (2005), Lippincott Williams & Wilkins (ISBN: 0781746736), incorporated herein by reference.

The compositions may further comprise additional actives, and in some embodiments, may further comprise contrast agents, including ultrasound and MRI contrast agents.

For intravenous infusion in various of the methods described below, dosages of adenosine and inosine are usefully established based upon the weight of the mammalian subject, such as a human patient, and dosages of each of the actives, adenosine and inosine, are usefully expressed as infusion rates given as μg/kg/min.

Thus, in embodiments intended for continuous intravenous infusion, adenosine is typically present in the pharmaceutical composition at a concentration, or in a weight amount, that permits adenosine to be infused at a rate between about 10 μg/kg/min to about 100 μg/kg/min.

In some of these embodiments, adenosine is present in an amount that permits infusion at a rate of at least about 10 μg/kg/min, at least about 15 μg/kg/min, at least about 20 μg/kg/min, at least about 25 μg/kg/min, at least about 30 μg/kg/min, at least about 35 μg/kg/min, at least about 40 μg/kg/min, at least about 45 μg/kg/min, at least about 50 μg/kg/min, at least about 55 μg/kg/min, at least about 60 μg/kg/min, at least about 65 μg/kg/min, at least about 70 μg/kg/min, at least about 75 μg/kg/min, at least about 80 μg/kg/min, at least about 85 μg/kg/min, at least about 90 μg/kg/min, at least about 95 μg/kg/min, and at least about 100 μg/kg/min.

In some embodiments, adenosine is present in the composition in an amount that permits infusion at a rate of no more than about 100 μg/kg/min, no more than about 95 μg/kg/min, no more than about 90 μg/kg/min, no more than about 85 μg/kg/min, no more than about 80 μg/kg/min, no more than about 75 μg/kg/min, no more than about 70 μg/kg/min, no more than about 65 μg/kg/min, no more than about 60 μg/kg/min, no more than about 55 μg/kg/min, no more than about 50 μg/kg/min, no more than about 45 μg/kg/min, no more than about 40 μg/kg/min, no more than about 35 μg/kg/min, no more than about 30 μg/kg/min, no more than about 25 μg/kg/min, no more than about 20 μg/kg/min, no more than about 15 μg/kg/min, and no more than about 10 μg/kg/min.

In embodiments intended for intra-arterial infusion, such as intracoronary infusion, or intra-atrial infusion, dosages are usefully set independently of the subject's size or circulatory volume. Thus, for intra-arterial (such as intracoronary) infusion, dosages are usefully expressed as infusion rates given as μg/min of each of the actives, adenosine and inosine.

In some of these embodiments, adenosine is present in the composition in an amount that permits intra-arterial infusion at a rate of at least about 10 μg/min, at least about 15 μg/min, at least about 20 μg/min, at least about 25 μg/min, at least about 30 μg/min, at least about 35 μg/min, at least about 40 μg/min, even at least about 45 μg/min.

In various embodiments, adenosine is present in the composition in an amount that permits adenosine to be infused at a rate of no more than about 45 μg/min, no more than about 40 μg/min no more than about 35 μg/min, no more than about 30 μg/min, no more than about 25 μg/min, no more than about 20 μg/min, no more than about 15 μg/min, even no more than 10 μg/min.

In embodiments intended for continuous intravenous infusion, inosine is typically present in the pharmaceutical composition at a concentration, or in a weight amount, that permits inosine to be infused at a rate between about 10 μg/kg/min to 600 μg/kg/min.

In some of these embodiments, inosine is present in an amount that permits infusion at a rate of at least about 10 μg/kg/min, at least about 20 μg/kg/min, at least about 30 μg/kg/min, at least about 40 μg/kg/min, at least about 50 μg/kg/min, at least about 60 μg/kg/min, at least about 70 μg/kg/min, at least about 80 μg/kg/min, at least about 90 μg/kg/min, at least about 100 μg/kg/min, at least about 110 μg/kg/min, at least about 120 μg/kg/min, at least about 130 μg/kg/min, at least about 140 μg/kg/min, at least about 150 μg/kg/min, at least about 160 μg/kg/min, at least about 170 μg/kg/min, at least about 180 μg/kg/min, at least about 190 μg/kg/min, at least about 200 μg/kg/min, and at least about 210 μg/kg/min, at least about 220 μg/kg/min, at least about 230 μg/kg/min, at least about 240 μg/kg/min, at least about 250 μg/kg/min, at least about 260 μg/kg/min, at least about 270 μg/kg/min, at least about 280 μg/kg/min, at least about 290 μg/kg/min, at least about 300 μg/kg/min, at least about 310 μg/kg/min, at least about 320 μg/kg/min, at least about 330 μg/kg/min, at least about 340 μg/kg/min, at least about 350 μg/kg/min, at least about 360 μg/kg/min, at least about 370 μg/kg/min, at least about 380 μg/kg/min, at least about 390 μg/kg/min, at least about 400 μg/kg/min, at least about 410 μg/kg/min, at least about 420 μg/kg/min, at least about 430 μg/kg/min, at least about 440 μg/kg/min, at least about 450 μg/kg/min, at least about 460 μg/kg/min, at least about 470 μg/kg/min, at least about 480 μg/kg/min, at least about 490 μg/kg/min, at least about 500 μg/kg/min, at least about 510 μg/kg/min, at least about 520 μg/kg/min, at least about 530 μg/kg/min, at least about 540 μg/kg/min, at least about 550 μg/kg/min, at least about 560 μg/kg/min, at least about 570 μg/kg/min, at least about 580 μg/kg/min, at least about 590 μg/kg/min, and at least about 600 μg/kg/min.

In some embodiments, inosine is present in the composition in an amount that permits intravenous infusion at a rate of no more than about 600 μg/kg/min, no more than about 590 μg/kg/min, no more than about 580 μg/kg/min, no more than about 570 μg/kg/min, no more than about 560 μg/kg/min, no more than about 550 μg/kg/min, no more than about 540 μg/kg/min, no more than about 530 μg/kg/min, no more than about 520 μg/kg/min, no more than about 510 μg/kg/min, no more than about 500 μg/kg/min, no more than about 490 μg/kg/min, no more than about 480 μg/kg/min, no more than about 470 μg/kg/min, no more than about 460 μg/kg/min, no more than about 450 μg/kg/min, no more than about 440 μg/kg/min, no more than about 430 μg/kg/min, no more than about 420 μg/kg/min, no more than about 410 μg/kg/min, of no more than about 400 μg/kg/min, no more than about 390 μg/kg/min, no more than about 380 μg/kg/min, no more than about 370 μg/kg/min, no more than about 360 μg/kg/min, no more than about 350 μg/kg/min, no more than about 345 μg/kg/min, no more than about 340 μg/kg/min, no more than about 335 μg/kg/min, no more than about 330 μg/kg/min, no more than about 325 μg/kg/min, no more than about 320 μg/kg/min, no more than about 315 μg/kg/min, no more than about 310 μg/kg/min, no more than about 305 μg/kg/min, no more than about 300 μg/kg/min, no more than about 295 μg/kg/min, no more than about 290 μg/kg/min, no more than about 285 μg/kg/min, no more than about 280 μg/kg/min, no more than about 275 μg/kg/min, no more than about 270 μg/kg/min, no more than about 265 μg/kg/min, no more than about 260 μg/kg/min, no more than about 255 μg/kg/min, no more than about 250 μg/kg/min, no more than about 245 μg/kg/min, no more than about 240 μg/kg/min, no more than about 235 μg/kg/min, no more than about 230 μg/kg/min, no more than about 225 μg/kg/min, no more than about 220 μg/kg/min, no more than about 215 μg/kg/min, no more than about 210 μg/kg/min, no more than about 200 μg/kg/min, no more than about 195 μg/kg/min, no more than about 190 μg/kg/min, no more than about 185 μg/kg/min, no more than about 180 μg/kg/min, no more than about 175 μg/kg/min, no more than about 170 μg/kg/min, no more than about 165 μg/kg/min, no more than about 160 μg/kg/min, no more than about 155 μg/kg/min, no more than about 150 μg/kg/min, no more than about 145 μg/kg/min, no more than about 140 μg/kg/min, no more than about 135 μg/kg/min, no more than about 130 μg/kg/min, no more than about 125 μg/kg/min, no more than about 120 μg/kg/min, no more than about 115 μg/kg/min, no more than about 110 μg/kg/min, no more than about 105 μg/kg/min, no more than about 100 μg/kg/min, no more than about 95 μg/kg/min, no more than about 90 μg/kg/min, no more than about 85 μg/kg/min, no more than about 80 μg/kg/min, no more than about 75 μg/kg/min, no more than about 70 μg/kg/min, no more than about 65 μg/kg/min, no more than about 60 μg/kg/min, no more than about 55 μg/kg/min, no more than about 50 μg/kg/min, no more than about 45 μg/kg/min, no more than about 40 μg/kg/min, no more than about 35 μg/kg/min, and no more than about 30 μg/kg/min, no more than about 25 μμg/kg/min, no more than about 20 μg/kg/min.no more than about 15 μg/kg/min, even no more than about 10 μg/kg/min.

In embodiments of the pharmaceutical composition intended for intra-arterial or intra-atrial infusion, inosine is typically present at a concentration, or in a weight amount, that permits inosine to be infused at a rate between about 20 μg/min to 100 μg/min.

In some of these embodiments, inosine is present in an amount that permits infusion at a rate of at least about 20 μg/min, at least about 30 μg/min, at least about 40 μg/min, at least about 50 μg/min, at least about 60 μg/min, at least about 70 μg/min, at least about 80 μg/min, at least about 90 μg/min, and at least about 100 μg/min.

In various embodiments, inosine is present in the composition in an amount that permits inosine to be infused at a rate of no more than about 100 μg/min, no more than about 95 μg/min, no more than about 90 μg/min, no more than about 85 μg/min, no more than about 80 μg/min, no more than about 75 μg/min, no more than about 70 μg/min, no more than about 65 μg/min, no more than about 60 μg/min, no more than about 55 μg/min, no more than about 50 μg/min, no more than about 45 μg/min, no more than about 40 μg/min, no more than about 35 μg/min, no more than about 30 μg/min, no more than about 25 μg/min, no more than about 20 μg/min.

5.3 Unit Dosage Forms and Kits

The pharmaceutical compositions of the present invention are usefully packaged in a unit dosage form that is adapted for the various clinical methods further described below.

In embodiments in which the pharmaceutical composition is in the form of a sterile, nonpyrogenic, liquid suitable for parenteral infusion, the composition may, for example, be packaged in volumes of 5-500 ml.

Convenient unit dosage forms for short intravenous infusion (e.g., for use in cardiac perfusion imaging, further described below) can contain 5 to 15 ml, typically 7, 10, 12 or 15 ml. Embodiments intended for longer intravenous infusion (e.g., for use in treating or prophylaxing against reperfusion injury), can contain 250, 300, 350, 400, 450, 500 ml, or more.

In certain embodiments, the unit dosage form contains, in a total of 10 ml, 30 mg of adenosine and 90 mg, 120 mg, 150 mg or 180 mg of inosine, with intermediate amounts of inosine permissible. In various embodiments, the unit dosage form contains, in a total of 250 ml, 750 mg adenosine with 75, 750, 1,500 or 2,250 mg of inosine, with intermediate inosine amounts permissible.

The container for unit dosage forms of the present invention are typically adapted for use with standard intravenous infusion sets.

Combined in a nonpyrogenic, fluid suitable for parenteral infusion, maximal adenosine solubility is about 4 mg/ml and that of inosine 20 mg/ml. Concentrations approaching one or both of these maxima are convenient for unit dosage forms intended for long infusion times, since high concentrations of the nucleoside actives reduce the volume required to be infused. Typical embodiments include, for example, 250 ml containers (vials, etc.) containing 1,000 mg of adenosine with 100 mg, 1,000 mg, 2,000 mg, or even 3,000 mg, 4,000 mg, 5,000 mg, even 6,000 mg of inosine, with intermediate inosine amounts permissible.

Even without optional preservatives, sterile-packaged compositions of the present invention may be stable at room temperature for at least one to two years.

In another aspect, kits are provided.

In one series of embodiments, the kit comprises a plurality of unit doses of the pharmaceutical composition of the present invention. In some embodiments, the plurality of unit doses have identical composition. In other embodiments, the composition among unit doses differs in amount and/or concentration of adenosine and/or inosine, or in the ratio therebetween. In certain embodiments, the kit further comprises an infusion set suitable for effecting intravenous infusion.

In other embodiments, kits comprise at least one unit dose of adenosine suitable for parenteral administration and at least one separately packaged unit dose of inosine suitable for parenteral administration. Usefully, the kit comprises a plurality of unit doses of adenosine, each having adenosine in a fixed amount or at a fixed concentration, packaged with a plurality of unit doses of inosine, at least a plurality of unit doses of inosine differing in the amount or concentration of inosine, permitting a desired A:I ratio readily to be selected. In certain embodiments, the kit further comprises an infusion set suitable for effecting intravenous infusion.

5.4 Cardioplegic Solutions

In another aspect, the invention provides cardioplegic solutions comprising adenosine and inosine at an A:I ratio of 1:1 to 1:20.

In various embodiments, the cardioplegic solutions comprise adenosine and inosine in an A:I ratio of at least about 1:1, 1:2, 1:3, 1:4, even at least about 1:5. In certain embodiments, the A:I ratio is usefully at least about 1:6, 1:7, 1:8, 1:9 or 1:10. In certain embodiments, the cardioplegic solution contains 0.5 to 3 mg/ml of adenosine and 0.5 to 15 mg/ml of inosine.

In some embodiments, the cardioplegic solution contains no additional cardioplegic actives, such as potassium. In other embodiments, adenosine and inosine are used to supplement existing cardioplegic solutions.

5.5 Methods

5.5.1 Administration

In the methods of the present invention, adenosine and inosine are administered concurrently by parenteral infusion in amounts, at A:I ratios, and for a time sufficient to achieve the desired therapeutic or diagnostic effect.

In some embodiments, adenosine and inosine may be administered as separate compositions. Such embodiments may be preferred when the A:I ratio is required or desired to be titrated or adjusted during the procedure. In these embodiments, compositions comprising adenosine and inosine may be infused into different vascular sites, or through the same site. Infusion through the same site can usefully be achieved by administration of the two compositions using a single infusion set. In other embodiments, adenosine and inosine are administered in a single composition having a defined A:I ratio, such as the pharmaceutical compositions above-described.

Programmable syringe pumps or micropumps, as are typical in clinical practice, are usefully employed to help avoid fluctuations of adenosine and inosine concentrations in plasma during the therapeutic or diagnostic method.

The route of administration is chosen based upon the desired clinical effect, as further described below. In certain embodiments, the pharmaceutical composition is administered by intravenous infusion. In other embodiments, the composition is administered by intra-arterial, such as intra-coronary, infusion. In yet other embodiments, the composition is administered by intra-atrial infusion. In certain embodiments, the composition is infused intrathecally. In yet other embodiments, the composition is administered as a perfusate.

In typical embodiments, other than for treatment of supraventricular tachycardia, bolus administration is disfavored, and adenosine and inosine are infused over a period of time of at least 1 minute, typically at least 2 minutes, 3 minutes, 4 minutes, 5 minutes, 6 minutes, and for various embodiments, even at least 10 minutes, 20 minutes, or 30 minutes. In certain embodiments, adenosine and inosine are infused for as long as 30 minutes, 40 minutes, 50 minutes, 60 minutes, even 120 minutes or more. As used herein, “continuous infusion” intends infusion over a period of at least 2 minutes.

In various embodiments, a first parenteral infusion may be followed by at least a second parenteral infusion, at the same or different dose of one or both of the actives.

In certain embodiments, the method comprises at least a first concurrent parenteral infusion of adenosine and inosine at an adenosine:inosine (A:I) weight ratio of about 1:1 to about 1:20. In certain embodiments, the ratio is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, even 1:20, with nonintegral ratios between 1:1 and 1:20 permissible. In certain presently preferred embodiments, the methods comprise concurrent infusion of adenosine and inosine at a ratio of about 1:2 to 1:10, preferably about 1:3 to about 1:6. For certain methods further described below, embodiments usefully comprise concurrent parenteral infusion of adenosine and inosine at an A:I weight ratio of about 1:4 or 1:5.

In certain embodiments, the methods comprise concurrent parenteral infusion of adenosine and inosine at A:I weight ratios of about 20:1 to about 1:1. In certain embodiments, the ratio is about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, even 1:1, with nonintegral ratios between 20:1 and 1:1 permissible. In certain of these embodiments, the A:I ratios are from about 2:1 to 10:1.

In some embodiments, adenosine is administered by intravenous infusion at an infusion rate between 10 μg/kg/min to 100 μg/kg/min. Thus, in some embodiments, adenosine is infused at a rate of at least about 10 μg/kg/min, at least about 15 μg/kg/min, at least about 20 μg/kg/min, at least about 25 μg/kg/min, at least about 30 μg/kg/min, at least about 35 μg/kg/min, at least about 40 μg/kg/min, at least about 45 μg/kg/min, at least about 50 μg/kg/min, at least about 55 μg/kg/min, at least about 60 μg/kg/min, at least about 65 μg/kg/min, at least about 70 μg/kg/min, at least about 75 μg/kg/min, at least about 80 μg/kg/min, at least about 85 μg/kg/min, at least about 90 μg/kg/min, at least about 95 μg/kg/min, and at least about 100 μg/kg/min.

In various embodiments, adenosine is infused intravenously at a rate of no more than about 100 μg/kg/min, no more than about 95 μg/kg/min, no more than about 90 μg/kg/min, no more than about 85 μg/kg/min, no more than about 80 μg/kg/min, no more than about 75 μg/kg/min, no more than about 70 μg/kg/min, no more than about 65 μg/kg/min, no more than about 60 μg/kg/min, no more than about 55 μg/kg/min, no more than about 50 μg/kg/min, no more than about 45 μg/kg/min, no more than about 40 μg/kg/min, no more than about 35 μg/kg/min, no more than about 30 μg/kg/min, no more than about 25 μg/kg/min, no more than about 20 μg/kg/min, no more than about 15 μg/kg/min, and no more than about 10 μg/kg/min.

In some embodiments, adenosine is administered by intra-arterial infusion, such as intracoronary infusion, at an infusion rate of 20 to 45 μg/min. Thus, in some embodiments, adenosine is infused at a rate of at least about 20 μg/min, at least about 25 μg/min, at least about 30 μg/min, at least about 35 μg/min at least about 40 μg/min, and at least about 45 μg/min.

In various embodiments, adenosine is infused intra-arterially at an infusion rate of no more than about 45 μg/min, no more than about 40 μg/min no more than about 35 μg/min, no more than about 30 μg/min, no more than about 25 μg/min, even no more than about 20 μg/min.

In some embodiments, inosine is administered by intravenous infusion at an infusion rate between 10 μg/kg/min to 600 μg/kg/min. Thus, in some embodiments, inosine is infused intravenously at a rate of at least about 10 μg/kg/min, at least about 20 μg/kg/min, at least about 30 μg/kg/min, at least about 40 μg/kg/min, at least about 50 μg/kg/min, at least about 60 μg/kg/min, at least about 70 μg/kg/min, at least about 80 μg/kg/min, at least about 90 μg/kg/min, at least about 100 μg/kg/min, at least about 110 μg/kg/min, at least about 120 μg/kg/min, at least about 130 μg/kg/min, at least about 140 μg/kg/min, at least about 150 μg/kg/min, at least about 160 μg/kg/min, at least about 170 μg/kg/min, at least about 180 μg/kg/min, at least about 190 μg/kg/min, at least about 200 μg/kg/min, and at least about 210 μg/kg/min, at least about 220 μg/kg/min, at least about 230 μg/kg/min, at least about 240 μg/kg/min, at least about 250 μg/kg/min, at least about 260 μg/kg/min, at least about 270 μg/kg/min, at least about 280 μg/kg/min, at least about 290 μg/kg/min, at least about 300 μg/kg/min, at least about 310 μg/kg/min, at least about 320 μg/kg/min, at least about 330 μg/kg/min, at least about 340 μg/kg/min, at least about 350 μg/kg/min, at least about 360 μg/kg/min, at least about 370 μg/kg/min, at least about 380 μg/kg/min, at least about 390 μg/kg/min, at least about 400 μg/kg/min, at least about 410 μg/kg/min, at least about 420 μg/kg/min, at least about 430 μg/kg/min, at least about 440 μg/kg/min, at least about 450 μg/kg/min, at least about 460 μg/kg/min, at least about 470 μg/kg/min, at least about 480 μg/kg/min, at least about 490 μg/kg/min, at least about 500 μg/kg/min, at least about 510 μg/kg/min, at least about 520 μg/kg/min, at least about 530 μg/kg/min, at least about 540 μg/kg/min, at least about 550 μg/kg/min, at least about 560 μg/kg/min, at least about 570 μg/kg/min, at least about 580 μg/kg/min, at least about 590 μg/kg/min. at least about 600 μg/kg/min.

In some embodiments, inosine is infused intravenously at a rate of no more than about 600 μg/kg/min, no more than about 590 μg/kg/min, no more than about 580 μg/kg/min, no more than about 570 μg/kg/min, no more than about 560 μg/kg/min, no more than about 550 μg/kg/min, no more than about 540 μg/kg/min, no more than about 530 μg/kg/min, no more than about 520 μg/kg/min, no more than about 510 μg/kg/min, no more than about 500 μg/kg/min, no more than about 490 μg/kg/min, no more than about 480 μg/kg/min, no more than about 470 μg/kg/min, no more than about 460 μg/kg/min, no more than about 450 μg/kg/min, no more than about 440 μg/kg/min, no more than about 430 μg/kg/min, no more than about 420 μg/kg/min, no more than about 410 μg/kg/min, of no more than about 400 μg/kg/min, no more than about 390 μg/kg/min, no more than about 380 μg/kg/min, no more than about 370 μg/kg/min, no more than about 360 μg/kg/min, no more than about 350 μg/kg/min, no more than about 345 μg/kg/min, no more than about 340 μg/kg/min, no more than 335 μg/kg/min, no more than about 330 μg/kg/min, no more than about 325 μg/kg/min, no more than about 320 μg/kg/min, no more than about 315 μg/kg/min, no more than about 310 μg/kg/min, no more than about 305 μg/kg/min no more than about 300 μg/kg/min, no more than about 295 μg/kg/min, no more than about 290 μg/kg/min, no more than about 285 μg/kg/min, no more than about 280 μg/kg/min, no more than about 275 μg/kg/min, no more than about 270 μg/kg/min, no more than about 265 μg/kg/min, no more than about 260 μg/kg/min, no more than about 255 μg/kg/min, no more than about 250 μg/kg/min, no more than about 245 μg/kg/min, no more than about 240 μg/kg/min, no more than about 235 μg/kg/min, no more than about 230 μg/kg/min, no more than about 225 μg/kg/min, no more than about 220 μg/kg/min, no more than about 215 μg/kg/min, no more than about 210 μg/kg/min, no more than about 205 μg/kg/min, no more than about 200 μg/kg/min, no more than about 195 μg/kg/min, no more than about 190 μg/kg/min, no more than about 185 μg/kg/min, no more than about 180 μg/kg/min, no more than about 175 μg/kg/min, no more than about 170 μg/kg/min, no more than about 165 μg/kg/min, no more than about 160 μg/kg/min, no more than about 155 μg/kg/min, no more than about 150 μg/kg/min, no more than about 145 μg/kg/min, no more than about 140 μg/kg/min, no more than about 135 μg/kg/min, no more than about 130 μg/kg/min, no more than about 125 μg/kg/min, no more than about 120 μg/kg/min, no more than about 115 μg/kg/min, no more than about 110 μg/kg/min, no more than about 105 μg/kg/min, no more than about 100 μg/kg/min, no more than about 95 μg/kg/min, no more than about 90 μg/kg/min, no more than about 85 μg/kg/min, no more than about 80 μg/kg/min, no more than about 75 μg/kg/min, no more than about 70 μg/kg/min, no more than about 65 μg/kg/min, no more than about 60 μg/kg/min, no more than about 55 μg/kg/min, no more than about 50 μg/kg/min, no more than about 45 μg/kg/min, no more than about 40 μg/kg/min, no more than about 35 μg/kg/min, no more than about 30 μg/kg/min, no more than 25 μg/kg/min, no more than 20 μg/kg/min, no more than 15 μg/kg/min and no more than 10 μg/kg/min.

In some embodiments, inosine is administered by intra-arterial infusion at an infusion rate between 20 μg/min to 100 μg/min. Thus, in some embodiments, inosine is infused intra-arterially at a rate of at least about 20 μg/min, at least about 30 μg/min, at least about 40 μg/min, at least about 50 μg/min,at least about 60 μg/min, at least about 70 μg/min, at least about 80 μg/min, at least about 90 μg/min, even at least about 100 μg/min.

In various embodiments, inosine is infused intra-arterially at an infusion rate of no more than about 100 μg/min, no more than about 95 μg/min, no more than about 90 μg/min, no more than about 85 μg/min, no more than about 80 μg/min, no more than about 75 μg/min, no more than about 70 μg/min, no more than about 65 μg/min, no more than about 60 μg/min, no more than about 55 μg/min, no more than about 50 μg/min, no more than about 45 μg/min, no more than about 40 μg/min, no more than about 35 μg/min, no more than about 30 μg/min, no more than about 25 μg/min, or no more than about 20 μg/min.

5.5.2 Pharmacological Stress Testing

In one aspect, the invention provides improved methods of pharmacologic stress testing, the improvement comprising the concurrent administration of adenosine and inosine as the pharmacologic stressor. Typically, adenosine and inosine are administered at an adenosine:inosine ratio of about 1:1 to about 1:20.

In certain embodiments, the adenosine:inosine ratio is at least about 1:3, 1:4, 1:5 or more. In various embodiments, the adenosine:inosine ratio is at least about 1:6, 1:7, 1:8, 1:9, 1:10, or even as high as at least about 1:20. In some embodiments, the adenosine:inosine ratio is no more than about 1:20, typically no more than about 1:15, no more than about 1:10, and may be no more than about 1:9, 1:8, 1:7, 1:6, 1:5, 1:4 or even no more than about 1:3.

Adenosine and inosine are infused at dosages sufficient to cause vasodilation of a coronary artery. In certain embodiments, adenosine and inosine are infused at dosages sufficient to cause maximal vasodilation of coronary arteries.

In typical embodiments, adenosine and inosine are infused at dosages that reduce the severity of or eliminate one or more of the side effects commonly seen when adenosine is used as single agent stressor at 140 μg/kg/min, such as hypotension; flushing; chest discomfort; dyspnea or urge to breathe deeply; headache; throat, neck or jaw discomfort; gastrointestinal discomfort; lightheadness/dizziness; upper extremity discomfort.

In certain embodiments, adenosine and inosine are administered by intravenous infusion. In typical embodiments, adenosine is continuously infused i.v. at a rate of less than about 140 μg/kg/min. In various embodiments, adenosine is infused at a rate of less than about 130 μg/kg/min, 120 μg/kg/min, 110 μg/kg/min, 100 μg/kg/min, even less than about 90 μg/kg/min, or less than about 80 μg/kg/min.

In certain embodiments, adenosine is infused at a rate of at least about 30 μg/kg/min, at least about 40 μg/kg/min, at least about 50 μg/kg/min, even at least about 60 or 70 μg/kg/min. In certain embodiments, adenosine is administered at an infusion rate of about 70 μg/kg/min, with inosine infused at an adenosine:inosine ratio of at least about 1:3, at least about 1:4, or at least about 1:5. In certain embodiments, adenosine is administered at a rate of about 70 μg/kg/min and inosine is infused at a rate of about 280 μg/kg/min. In other embodiments, adenosine is infused at a rate of 50-70 μg/kg/min with inosine 150-420 μg/kg/min (A:I ratio of 1:3 to 1:6).

Conveniently, adenosine and inosine can be administered by continuous peripheral intravenous infusion of a pharmaceutical composition of the present invention comprising both adenosine and inosine, as described hereinabove.

The efficacy of BIDOSINE permits shorter infusion times as compared to use of adenosine as single agent. In some embodiments, adenosine and inosine are infused for a period of at least 6 minutes. In other embodiments, adenosine and inosine are infused continuously for a period shorter than 6 minutes, including infusions of 5 minutes, 4 minutes, 3 minutes, and even 2 minutes. Short infusion times present advantages in reducing the frequency and magnitude of side effects.

Accordingly, the pharmacologic stress test methods described herein further comprise the step of qualitatively or quantitatively assessing one or more parameters of cardiac function during the infusion. Functions usefully measured include, in various embodiments, imaging of myocardial perfusion, imaging or measurement of ventricular function, and measuring coronary blood flow velocity.

In various embodiments, assessment of cardiac function includes use of one or more techniques selected from the group consisting of: electrocardiography, echography (M mode, two and three dimensional), echo-doppler, cardiac imaging, including planar (conventional) scintigraphy, single photon emission computed tomography (SPECT), dynamic single photon emission computed tomography (D-SPECT™ Cardiac Scan), positron emission tomography (PET), radionuclide angiography (first pass and equilibrium studies utilizing, e.g., technetium 99m-labeled red blood cells), nuclear magnetic resonance (NMR) imaging, perfusion contrast echocardiography, digital subtraction angiography (DSA), and ultrafast x-ray computed tomography (CINE CT). SPECT studies can be performed using any of the isotopes known to be suitable for such studies, such as thallium-201, technetium sestamibi, tetrofosmine. PET studies can be performed using any of the isotopes known to be suitable for such studies, such as rubidium 82, nitrogen-13, fluorine-18, carbon-11, Boron-11, and oxygen-15.

Typically, isotope is injected during the infusion of adenosine with inosine, and imaging begins after the end of the infusion.

5.5.3 Treatment of Myocardial Reperfusion Injury

As demonstrated in Example 3, below, concurrent parenteral infusion of adenosine and inosine preserves ejection fraction when administered shortly after cardiac ischemic insult in an experimental animal model of acute coronary insufficiency.

Accordingly, in another aspect, the invention provides methods of treating post-ischemic myocardial injury, such as reperfusion injuries. Treating includes prophylaxis, and includes reduction in the severity, size, and/or symptoms of post-ischemic reperfusion injury, as well as restoration of cardiac function.

The methods comprise administering at least a first concurrent parenteral infusion of adenosine and inosine, the adenosine and inosine being infused at an A:I ratio of about 1:1 to about 1:20, during or following an acute ischemic incident. The acute ischemic incident may, for example, be a myocardial infarction.

In typical embodiments, adenosine and inosine are infused intravenously. In some embodiments, adenosine and inosine are infused in a single composition comprising both nucleosides, such as the pharmaceutical compositions above-described.

In various embodiments, adenosine is infused at a rate of less than about 140 μg/kg/min. In some embodiments, adenosine is infused at a rate of less than 130 μg/kg/min, less than 120 μg/kg/min, less than 110 μg/kg/min, even less than 100 μg/kg/min. In certain embodiments, adenosine is infused at a rate of less than 90 μg/kg/min, even less than about 80 μg/kg/min. In some embodiments, adenosine is infused at a rate of 50-70 μg/kg/min, including nonintegral values therebetween.

In typical embodiments, the A:I ratio is between 1:1 to 1:20. In certain embodiments, the ratio is at least 1:1, at least 1:2, at least 1:3, at least 1:4, even at least 1:5, 1:6, 1:7, 1:8, 1:9, or 1:10. In some embodiments, the ratio is less than about 1:20, 1:19, 1:18, 1:17, 1:16, or 1:15. In certain embodiments, adenosine is infused at a rate of 50-70 μg/kg/min and inosine is usefully infused at a rate of 50-210 μg/kg/min (A:I ratio of 1:1 to 1:3). In other embodiments, adenosine is infused at a rate of 20-30 μg/kg/min with inosine infused at a rate of 200-600 μg/kg/min (A:I ratio of 1:10 to 1:20).

The first parenteral infusion is preferably begun as soon as ischemia is detected, and may usefully be initiated as late as 24-48 hours after ischemic insult.

In certain embodiments, the method further comprises at least one subsequent parenteral infusion of adenosine and inosine at an A:I ratio of 1:1 to 1:20. In some embodiments, the one or more subsequent infusions is at the same adenosine and inosine infusion rate and A:I ratio as the first infusion. In other embodiments, one or more subsequent infusions differs from the first infusion in one or more of adenosine infusion rate, inosine infusion rate, or A:I ratio.

In various embodiments, each of the first and optional subsequent infusions is for a period of at least about 10 minutes. In typical embodiments, each infusion is for a period of at least about 20 minutes, 30 minutes, 40 minutes, 50 minutes, even at least about 60 minutes, 120 minutes, or even at least about 180 minutes. Usefully, each infusion is about 60-120 minutes in duration. In certain embodiments, infusions are administered once a day, twice a day, three times a day, even 4 or more times a day, typically for at least one day, 2 days, 3 days, even as much as 5 days, after detection of ischemia. Usefully, infusions can be administered for the duration of a patient's in-hospital stay.

The preferred dosages of adenosine and inosine for any individual patient can readily be determined by those skilled in cardiology by individually titrating the adenosine and/or inosine infusion rates to achieve a desired level of improvement in one or more hemodynamic parameters, such as coronary blood flow, cardiac output, myocardial perfusion, or left ventricular ejection fraction. In addition or in the alternative, adenosine infusion rate can first be titrated to a maximum dosage that does not occasion significant hypotension; flushing; chest discomfort; dyspnea or urge to breathe deeply; headache; throat, neck or jaw discomfort; gastrointestinal discomfort; lightheadedness/dizziness; or upper extremity discomfort, and inosine dosage then individually titrated to achieve desired hemodynamic parameters, such as a desired left ventricular systolic pressure, mean arterial blood pressure, cardiac output, coronary blood flow, myocardial perfusion, or left ventricular ejection fraction.

5.5.4 Increase in Myocardial Microcirculation

Coadministration of adenosine and inosine exert effects on the myocardial microcirculation, as well as on the coronary arteries.

Accordingly, in another aspect, the invention provides methods of increasing myocardial microcirculation, e.g., following acute myocardial infarct, chronic ischemia, and/or congestive heart failure, the methods comprising concurrent administration of adenosine and inosine by parenteral infusion, with or without additional therapeutic agents.

The methods comprise administering at least a first concurrent parenteral infusion of adenosine and inosine, the adenosine and inosine being infused at an A:I ratio of about 1:1 to about 1:20, to a patient having at least one region of myocardial ischemia, including one or more regions of myocardial infarct.

In typical embodiments, adenosine and inosine are infused intravenously. In some embodiments, adenosine and inosine are infused in a single composition comprising both nucleosides, such as the pharmaceutical compositions above-described.

In various embodiments, adenosine is infused at a rate of less than about 140 μg/kg/min. In some embodiments, adenosine is infused at a rate of less than 130 μg/kg/min, less than 120 μg/kg/min, less than 110 μg/kg/min, even less than 100 μg/kg/min. In certain embodiments, adenosine is infused at a rate of less than 90 μg/kg/min, even less than about 80 μg/kg/min. In some embodiments, adenosine is infused at a rate of 50-70 μg/kg/min, including nonintegral values therebetween.

In typical embodiments, the A:I ratio is between 1:1 to 1:20. In certain embodiments, the ratio is at least 1:2, at least 1:3, at least 1:4, even at least 1:5, 1:6, 1:7, 1:8, 1:9, or 1:10. In some embodiments, the ratio is less than about 1:20, 1:19, 1:18, 1:17, 1:16, or 1:15. In certain embodiments, adenosine is infused at a rate of 50-70 μg/kg/min and inosine is usefully infused at a rate of 50-210 μg/kg/min (A:I ratio of 1:1 to 1:3). In other embodiments, adenosine is infused at a rate of 20-30 μg/kg/min with inosine infused at a rate of 200-600 μg/kg/min (A:I ratio of 1:10 to 1:20).

In alternative embodiments, adenosine and inosine are administered by direct intracoronary infusion. In certain such embodiments, adenosine is infused at 20 -40 μg/min, with inosine usefully infused at 20-100 μg/min.

The first parenteral infusion is usefully begun as soon as ischemia is detected, but may be initiated after the acute ischemic insult has resolved.

In certain embodiments, the method further comprises at least one subsequent parenteral infusion of adenosine and inosine at an A:I ratio of 1:1 to 1:20. In some embodiments, the one or more subsequent infusions is at the same adenosine and inosine infusion rate and A:I ratio as the first infusion. In other embodiments, one or more subsequent infusions differs from the first infusion in one or more of adenosine infusion rate, inosine infusion rate, or A:I ratio.

In various embodiments, each of the first and optional subsequent infusions is for a period of at least about 5 minutes. In typical embodiments, each infusion is for a period of at least about 10 minutes, at least about 20 minutes, 30 minutes, 40 minutes, 50 minutes, 60 minutes, even at least about 120 minutes. Usefully, each infusion is about 30-60 minutes in duration. In certain embodiments, infusions are administered once a day, twice a day, three times a day, even 4 or more times a day, typically for at least one day, 2 days, 3 days, even as much as 5 days.

The preferred dosages of adenosine and inosine for any individual patient can readily be determined by those skilled in cardiology by individually titrating the adenosine and/or inosine infusion rates to achieve a desired level of improvement in one or more hemodynamic parameters, such as coronary blood flow, cardiac output, myocardial perfusion, or left ventricular ejection fraction. In addition or in the alternative, adenosine infusion rate can first be titrated to a maximum dosage that does not occasion significant hypotension; flushing; chest discomfort; dyspnea or urge to breathe deeply; headache; throat, neck or jaw discomfort; gastrointestinal discomfort; lightheadedness/dizziness; or upper extremity discomfort, and inosine dosage then individually titrated to achieve desired hemodynamic parameters, such as a desired left ventricular systolic pressure, mean arterial blood pressure, cardiac output, coronary blood flow, myocardial perfusion, or left ventricular ejection fraction.

5.5.5 Treatment of Nervous System Injury

As demonstrated in the Examples below, concurrent parenteral administration of adenosine and inosine hastens behavioral recovery in a standard animal model of acute ischemic spinal cord injury.

Accordingly, in another aspect the invention provides methods of treating injury to the central or peripheral nervous system by concurrent parenteral administration of adenosine and inosine at A:I ratios of about 1:1 to about 1:5, or about 1:1 to 10:1.

In various embodiments, the injury is acute and spontaneous, e.g. an acute insult of vascular origin, such as stroke, or an acute spinal cord injury. In certain embodiments, the injury is acute and of nosocomial origin, arising e.g. from neurosurgery, including e.g. spinal surgery, such as spinal surgery for relief of cord or root compression, and brain surgery, such as surgery for cerebral aneurysm. In some embodiments, no acute injury is detectable, and the methods are applied prophylactically or adjunctively in a surgical or medical setting that is known to be associated with injury to the nervous system, whether induced by ischemia or by emboli, including, e.g., carotid artery surgery; carotid endarterectomy; cardiac surgery, including but not exclusively heart surgery with cardiopulmonary bypass; surgery on the spine, including relief of cord/root compression; neurosurgical procedure on the brain, including cerebral aneurysm surgery;

In typical embodiments, adenosine and inosine are infused intravenously. In some embodiments, adenosine and inosine are infused in a single composition comprising both nucleosides, such as the pharmaceutical compositions above-described.

In various embodiments, adenosine is infused at a rate of less than about 140 μg/kg/min. In some embodiments, adenosine is infused at a rate of less than 130 μg/kg/min, less than 120 μg/kg/min, less than 110 μg/kg/min, even less than 100 μg/kg/min. In certain embodiments, adenosine is infused at a rate of less than 90 μg/kg/min, even less than about 80 μg/kg/min. In some embodiments, adenosine is infused at a rate of 35-70 μg/kg/min, including nonintegral values therebetween.

In typical embodiments, the A:I ratio is between 1:1 to 1:5. In certain embodiments, the ratio is at least 1:2, at least 1:3, at least 1:4, even at least 1:5. In some embodiments, the ratio is conversely about 10:1, 9:1, 8:1, 7:1, 6:1, 5:1, 4:1, 3:1, or 2:1, with nonintegral ratios permissible. In certain embodiments, adenosine is infused at a rate of adenosine 35-70 μg/kg/min with inosine infused at 10-210 μg/kg/min (maximal A:I ratio of 1:3). In other embodiments, adenosine is infused at 50-70 μg/kg/min with inosine at 10 to 35 μg/kg/min. In certain intraoperative embodiments, adenosine is administered intravenously during surgery at 70-100 μg/kg/min, with concurrent infusion of inosine at 10-500 μg/kg/min.

In the case of acute injury, the first parenteral infusion is preferably begun as soon as injury is detected, preferably no later than 6 hours after onset of injury. Thus, in some embodiments, a first parenteral infusion is initiated within 1 hour following injury, 2 hours following injury, 3 hours following injury, 4 hours following injury, even 5 or 6 hours following injury, although later initiation of a first infusion finds use in certain embodiments, including those in which injury is ongoing.

In embodiments in which treatment is prophylactic or adjunctive to surgery, the first parenteral infusion is usefully begun during surgery. In various surgical embodiments, adenosine and inosine are administered in addition or in the alternative by parenteral infusion shortly after the end of the surgical procedure, during the intensive care unit period. In certain post-surgical embodiments, adenosine is infused intravenously at a rate of no more than about 70 μg/kg/min, in some embodiments no more than about 50 μg/kg/min, and inosine is infused intravenously at an infusion rate of no more than about 210 μg/kg/min and sometimes no more than about 10 μg/kg/min. In certain post-surgical embodiments, the A:I ratio ranges from 1:1 to 7:1 or from 10:1 to 4:1.

In certain embodiments, the method further comprises at least one subsequent parenteral infusion of adenosine and inosine at an A:I ratio of 1:1 to 1:20. In some embodiments, the one or more subsequent infusions is at the same adenosine and inosine infusion rate and A:I ratio as the first infusion. In other embodiments, one or more subsequent infusions differs from the first infusion in one or more of adenosine infusion rate, inosine infusion rate, or A:I ratio.

In various embodiments, each of the first and optional subsequent infusions is for a period of at least about 10 minutes. In typical embodiments, each infusion is for a period of at least about 20 minutes, 30 minutes, 40 minutes, 50 minutes, 60 minutes, even at least about 120 and 180 minutes. Usefully, each infusion is about 60-120 minutes in duration. In certain embodiments, infusions are administered once a day, twice a day, three times a day, even 4 or more times a day, up to 12 times a day, typically for at least one day, 2 days, 3 days, even as much as 5 days or 10 days, after injury or detection of ischemia. In some embodiments, infusions are usefully discontinued after 3 days, 4 days, 5 days, post-injury. Usefully, infusions can be administered for the duration of a patient's in-hospital stay.

In other embodiments, adenosine and inosine are administered concurrently by intrathecal infusion at A:I ratios of about 1:1 to about 1:20. In other embodiments, administration is by infusion into cerebrospinal fluid. In certain of these embodiments, infusion rates of adenosine and inosine are titrated to provide a local concentration of adenosine at the site of injury of 5 to 25 μM, with a local concentration of inosine at the site of injury from 25 to 50 μM.

The preferred dosages of adenosine and inosine for any individual patient can readily be determined by those skilled in the clinical arts by individually titrating the adenosine and/or inosine infusion rates upwards to levels that do not adversely affect systemic blood pressure and/or cerebral blood flow. For example, in embodiments in which adenosine and inosine are administered concurrently by parenteral infusion postoperatively to a neurosurgery patient, the dosages of adenosine and/or inosine can be titrated to maintain systolic blood pressure within 20% of pre-surgical steady state.

Because adenosine can be used efficaciously at dosages that do not cause significant patient discomfort, the methods herein presented can be used in conscious patients.

5.5.6 Treatment of Acute Pulmonary Vascular Resistance

In another aspect, methods are provided for treating acute pulmonary vascular resistance by at least a first concurrent parenteral administration of adenosine and inosine at A:I ratios of about 1:1 to about 1:20, including acute pulmonary resistance during sudden and severe right ventricular dysfunction, and acute collapse associated with chronic pulmonary diseases and respiratory distress syndromes.

In typical embodiments, adenosine and inosine are infused intravenously. In other embodiments, adenosine and inosine are infused intra-arterially, such as directly into the pulmonary artery. In some embodiments, adenosine and inosine are infused in a single composition comprising both nucleosides, such as the pharmaceutical compositions above-described.

In various embodiments, adenosine is infused intravenously at a rate of less than about 140 μg/kg/min. In some embodiments, adenosine is infused at a rate of less than 130 μg/kg/min, less than 120 μg/kg/min, less than 110 μg/kg/min, even less than 100 μg/kg/min. In certain embodiments, adenosine is infused at a rate of less than 90 μg/kg/min, even less than about 80 μg/kg/min. In some embodiments, adenosine is infused at a rate of 30-70 μg/kg/min, including nonintegral values therebetween.

In typical embodiments, the A:I ratio is between 1:1 to 1:20. In certain embodiments, the ratio is at least 1:2, at least 1:3, at least 1:4, even at least 1:5, 1:6, 1:7, 1:8, 1:9, or 1:10. In some embodiments, the ratio is less than about 1:20, 1:19, 1:18, 1:17, 1:16, or 1:15.

In certain intravenous infusion embodiments, adenosine is infused intravenously at a rate of 30 μg/kg/min and inosine is infused at a rate of 90-150 μg/kg/min (A:I ratio of 1:3 to 1:5). In another embodiment, adenosine is infused intravenously at 50 μg/kg/min with inosine concurrently infused at 250 μg/kg/min (molar ratio 1:5). In yet other embodiments, adenosine and inosine are both infused at 50 μg/kg/min (an A:I ratio of 1:1).

The first parenteral infusion is preferably begun as soon increased pulmonary vascular resistance is detected.

In certain embodiments, the method further comprises at least one subsequent parenteral infusion of adenosine and inosine at an A:I ratio of 1:1 to 1:20. In some embodiments, the one or more subsequent infusions is at the same adenosine and inosine infusion rate and A:I ratio as the first infusion. In other embodiments, one or more subsequent infusions differs from the first infusion in one or more of adenosine infusion rate, inosine infusion rate, or A:I ratio.

5.5.7 Adjunct to Cardiac Revascularization Procedures

In another aspect, methods are provided for improving clinical outcomes in percutaneous transluminal coronary angioplasty (PTCA) and/or coronary thrombolysis procedures, with or without stent placement, including procedures conducted in patients with acute coronary syndrome.

In various embodiments, adenosine and inosine are infused at an A:I ratio of from 1:1 to 1:20, including ratios of 1:1 to 1:10. In other embodiments, the ratio is usefully 7:1 to 5:1.

In various embodiments, adenosine is usefully administered intraoperatively during PTCA and/or coronary thrombolysis, and optionally post-operatively, by intravenous infusion at 35-70 μg/kg/min, with inosine at 35-70 μg/kg/min (A:I ratio 1:1). In other embodiments, adenosine is infused at 35 μg/kg/min with inosine at 35 μg/kg/min (A:I of 1:1), and in yet other embodiments, adenosine is infused at 50 μg/kg/min with inosine at 50 μg/kg/min. In some embodiments, adenosine is infused at 20-30 μg/kg/min with inosine concurrently infused at 200-600 μg/kg/min. In yet other embodiments, adenosine is administered intravenously at 50-701μg/kg/min with inosine at 10 μg/kg/min (A:I ratios of 7:1 to 5:1). In alternative embodiments, adenosine and inosine are administered by direct intracoronary infusion. In certain such embodiments, adenosine is infused at 20-40 μg/min, with inosine usefully infused at 20-100 μg/min.

In typical embodiments, administration is begun a few minutes before and is continued during the revascularization procedure, and can be continued for several hours.

In certain embodiments, thrombolytic agents, such as streptokinase, urokinase and tissue plasminogen activator, are coadministered with one or both of adenosine and inosine. In certain embodiments, adenosine and inosine are concurrently administered in a single composition, such as the pharmaceutical compositions above-described, with one or more thrombolytic agents concurrently administered in a second composition, or admixed therein.

5.5.8 Increased Cardiac Output

In one aspect, adenosine and inosine are concurrently infused to increase cardiac output, typically in medical conditions in which such increase is desired to be achieved without increase in cardiac work. In some embodiments, for example, adenosine and inosine are administered concurrently to treat acute heart failure (cardiogenic shock), with or without further administration of dopamine. In various embodiments, adenosine is infused intravenously at a rate of 35-50 μg/kg/min with inosine at a rate of 35-150 μg/kg/min (A:I ratio of 1:1 to 1:3). In some embodiments, in which long infusion times (one to several hours) may be required to stabilize cardiac performance, to reduce the risk of side effects due to adenosine accumulation, A:I ratios of 1:10 to 1:20 may be preferred. For example, in certain embodiments, adenosine is infused intravenously at 20-30 μg/kg/min with inosine at 200-600 μg/kg/min.

5.5.9 Intraoperative and Postoperative Analgesia

In another aspect, methods are provided for reducing intraoperative and postoperative pain, by concurrent parenteral infusion of adenosine and inosine at ratios of about 1:1 to about 1:20. In certain embodiments, adenosine is infused intravenously at 70-100 μg/kg/min with inosine at 70-210 μg/kg/min.

In another aspect, concurrent parenteral administration of adenosine and inosine in ratios of about 1:1 to about 1:20, or in other embodiments, at ratios of 7:1 to 5:1, is used to reduce postoperative complications in patients undergoing coronary artery bypass surgery. In certain of these embodiments, adenosine is administered by intravenous infusion at 70-100 μg/kg/min with inosine at 10-500 μg/kg/min combination. In some embodiments, further administration of adenosine and inosine is effected by incorporation of the nucleosides in a cardioplegia solution, as above-described, during the clamping period.

### 6. EXAMPLES

### Example 1

### Inosine Potentiates Adenosine Effects on Arterial Blood Flow in Rats

A study was conducted to determine whether, and at what dose, inosine might in combination sufficiently potentiate the effects of adenosine on arterial blood flow to permit adenosine to be administered with maximal efficacy at half the dose that is required to achieve maximal effect when adenosine is administered as a single agent (“adenosine max”, or “adenosine max dose”).

The study was conducted on 7 week old male Wistar rats weighing 250-300 g. Rats were anaesthetized with Inactin 100 mg/kg i.p. Inactin was preferred to other anaesthetics because of its moderate impact on blood pressure. Heparin-filled (100 IU/ml in saline) catheters were introduced into the left common carotid artery and into the left jugular vein for measurement of blood pressure and drug administration respectively. A flow probe was inserted around the right carotid artery for measurement of carotid blood flow (CaBF).

After completion of the surgical procedure, an equilibration period of 15 minutes was observed to obtain stable hemodynamic conditions.

In a first series of experiments, adenosine was administered as a single agent to identify the dose providing maximal increase in carotid arterial blood flow (“adenosine max”). In this experiment, various concentrations of adenosine were infused continuously through the jugular vein catheter for 5 minutes, with concurrent measurement of carotid arterial blood flow.

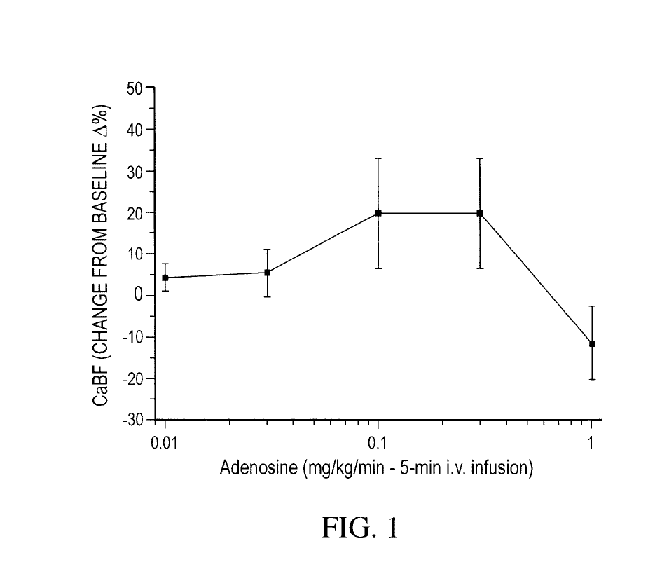

Results are summarized in FIG. 1. Data show the effects of increasing doses (each administered as a 5 min continuous i.v. infusion) of adenosine (0.01, 0.03, 0.1, 0.3 and 1 mg/kg/min) on CaBF (mL/min) in the anaesthetized rat. Values are expressed as percentage variations from baseline CaBF (Mean±SEM, n=6). The data demonstrate that the adenosine dose providing maximal CaBF as a single agent in this system is 0.1 mg/kg/min.

A second series of experiments was performed to assess whether inosine could potentiate the effects on CaBF of a continuous intravenous infusion of adenosine at half adenosine max.

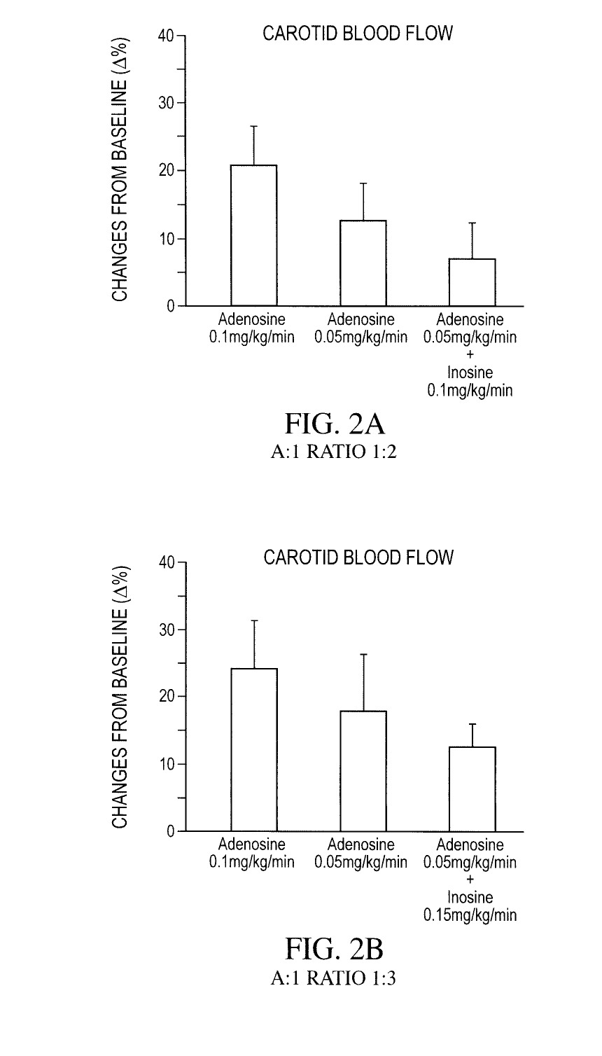

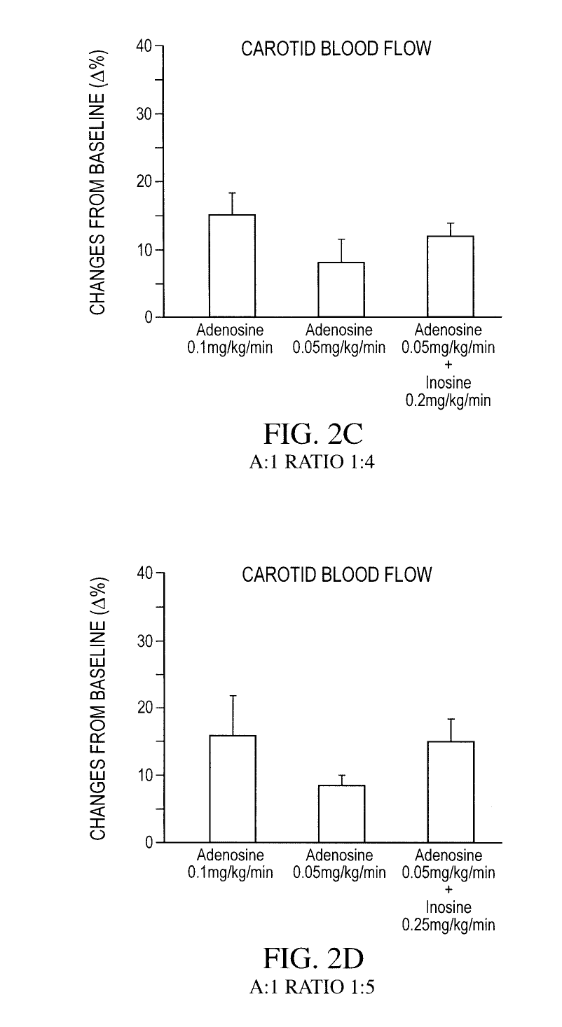

In each experiment, the effect on CaBF of (i) adenosine as a single agent, infused continuously for five minutes at adenosine max (0.1 mg/kg/min), (ii) adenosine as single agent infused continuously for five minutes at half adenosine max (0.05 mg/kg/min), and (iii) a composition containing a fixed dose of adenosine at half adenosine max (0.05 mg/kg/min) combined with inosine infused continuously for five minutes, was assessed. In separate experiments, inosine was included in the combined (“BIDOSINE”) composition in amounts required to achieve infusion at 0.1 mg/kg/min (A:I weight ratio of 1:2, FIG. 2A), 0.15 mg/kg/min (A:I weight ratio of 1:3, FIG. 2B), 0.2 mg/kg/min (A:I weight ratio of 1:4, FIG. 2C), and 0.25 mg/kg/min (A:I weight ratio of 1:5, FIG. 2D).

As seen in FIGS. 2A-2D, inosine potentiates the ability of adenosine to increase CaBF at A:I ratios of 1:4 and 1:5.

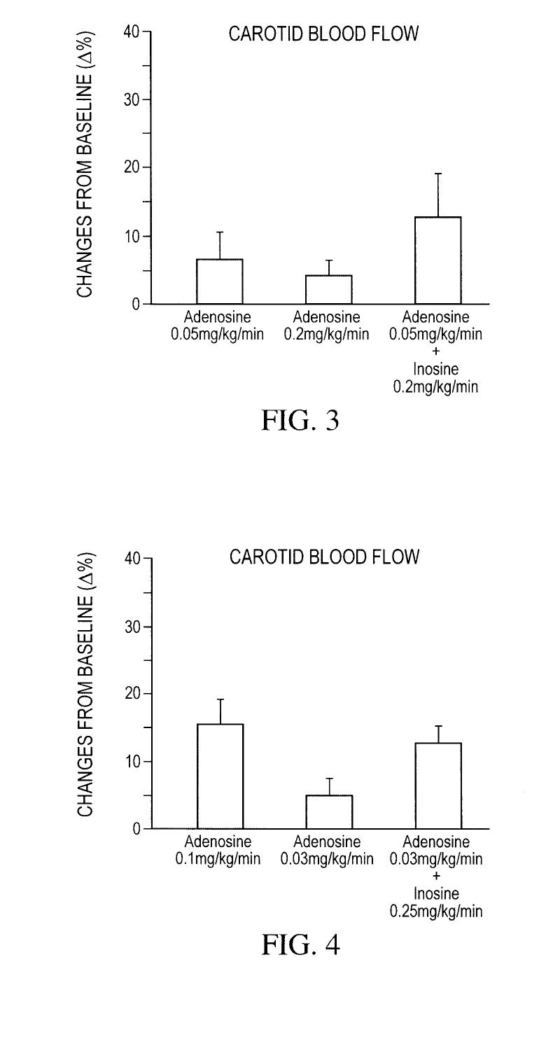

FIG. 3 shows the synergistic effect on increase in CaBF of combining adenosine at half adenosine max with inosine at an A:I weight ratio of 1:4. Data show the effects on CaBF (mL/min) of 6 minute continuous i.v. infusions of adenosine (0.05 mg/kg/min), inosine (0.2 mg/kg/min), and adenosine (0.05 mg/kg/min)+inosine (0.2 mg/kg/min), on CaBF (mL/min) in anaesthetized rats. Values are expressed as percentage variations from baseline (Mean±SEM, n=6). The data demonstrate that combination of adenosine at half adenosine max (0.05 mg/kg/min) with inosine at 0.2 mg/kg/min (A:I weight ratio of 1:4) increased CaBF by 13±6%, as compared to an increase of 7±4% with adenosine alone at adenosine half max (0.05 mg/kg/min), and as compared to an increase of 4±2% with inosine alone at 0.2 mg/kg/min. Expressed as percentage increase from baseline, the increase in CaBF effected by the combination was greater than the sum of increases caused by adenosine and inosine as single agents.

In another series of experiments, the adenosine fixed dose was slightly reduced from half adenosine max of 0.05 mg/kg/min, to 0.03 mg/kg/min and the inosine dose was slightly augmented to 0.25 mg/kg/min, to provide an A:I weight ratio of 1:8 ratio. Results are shown in FIG. 4.

FIG. 4 shows that the potentiating effect of combining adenosine with inosine is still seen when adenosine doses are reduced below half adenosine max. The data show the effects on CaBF (mL/min) of 6 minute continuous i.v. infusions of adenosine (0.1 mg/kg/min, adenosine max), adenosine (0.03 mg/kg/min), and adenosine (0.03 mg/kg/min)+inosine (0.25 mg/kg/min) in anaesthetized rats. In this example, potentiation is seen at an adenosine dose of 0.03 mg/kg/min, combined with inosine at 0.25 mg/kg/min, an A:I weight ratio of 1:8. Even at this reduced adenosine posology, the effect on CaBF is clearly potentiated by inosine. Values are expressed as percentage change from baseline (Mean±SEM, n=6).

Inosine alone does not increase CaBF in this system.

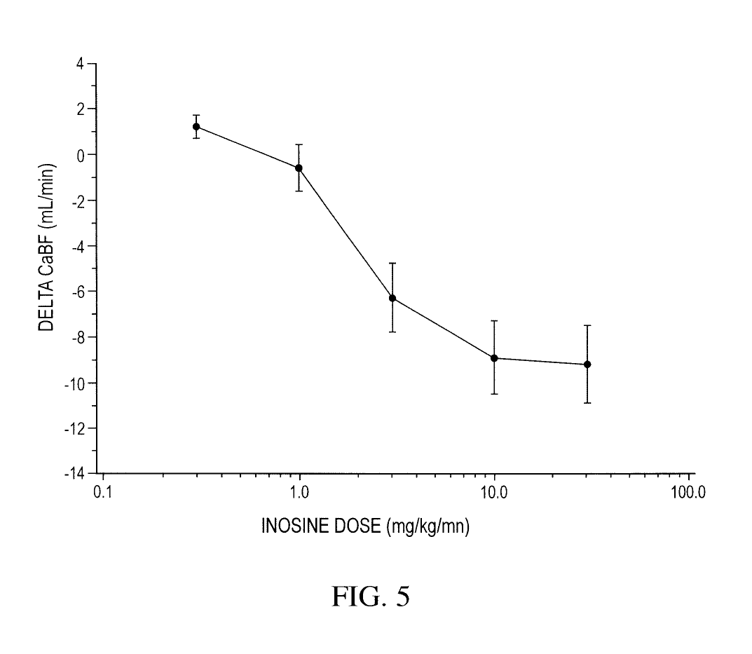

FIG. 5 shows that inosine alone is ineffective at increasing CaBF, even at doses 10 times those at which adenosine alone is effective. Data show the effects on CaBF (mL/min) of increasing doses of inosine (0.3, 1, 3, 10 and 30 mg/kg/min), each administered as a 5 minute i.v. infusion, in anaesthetized rats. Values are expressed as absolute variations from baseline, in mL/min (Mean±SEM, n=6). No response is seen at 0.3 mg/kg/min. Modest decreases in CaBF are seen at 1 mg/kg/min (10 times adenosine max). Between 1 to 10 mg/kg/min, there is a sharp slump and dose-dependent decrease in CaBF.

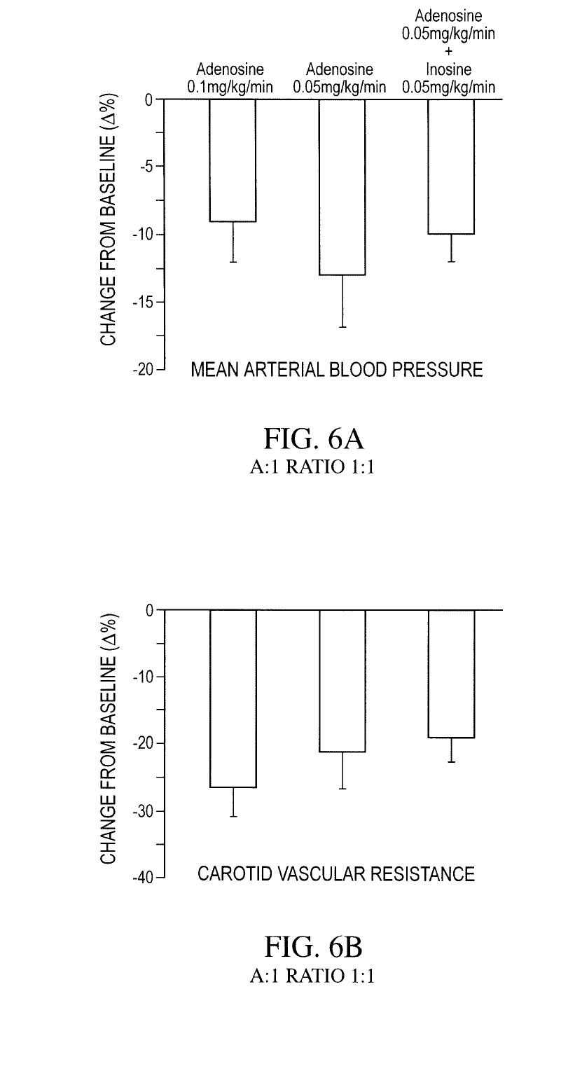

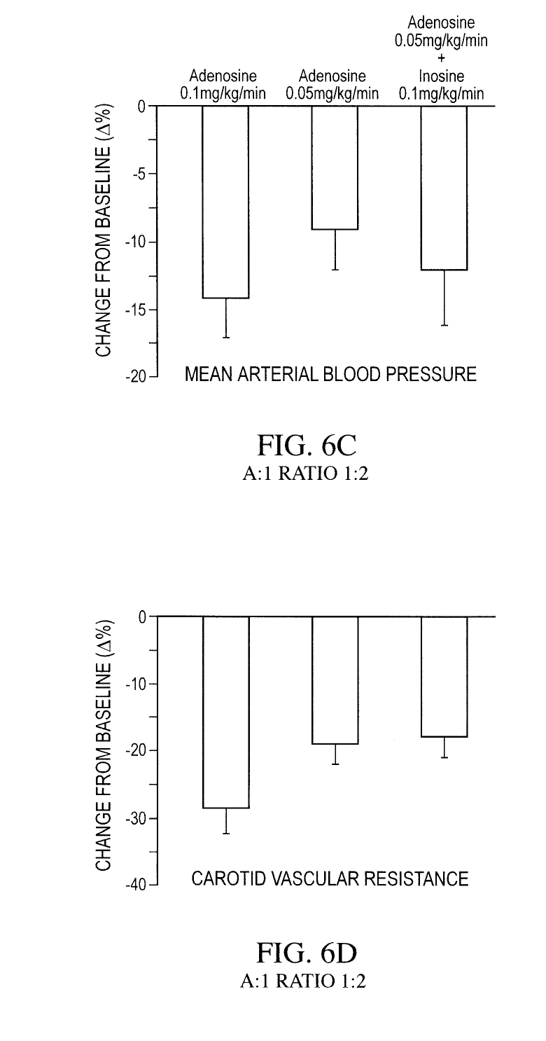

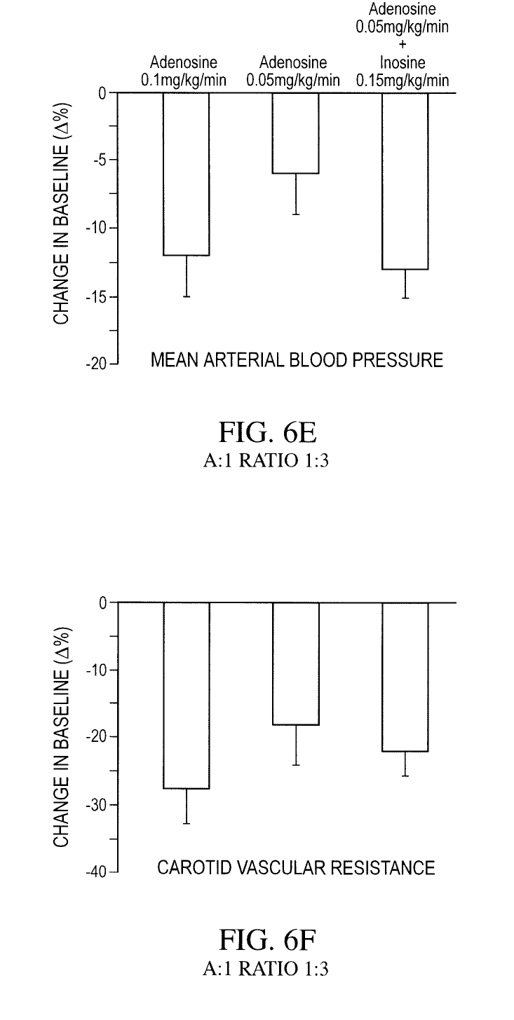

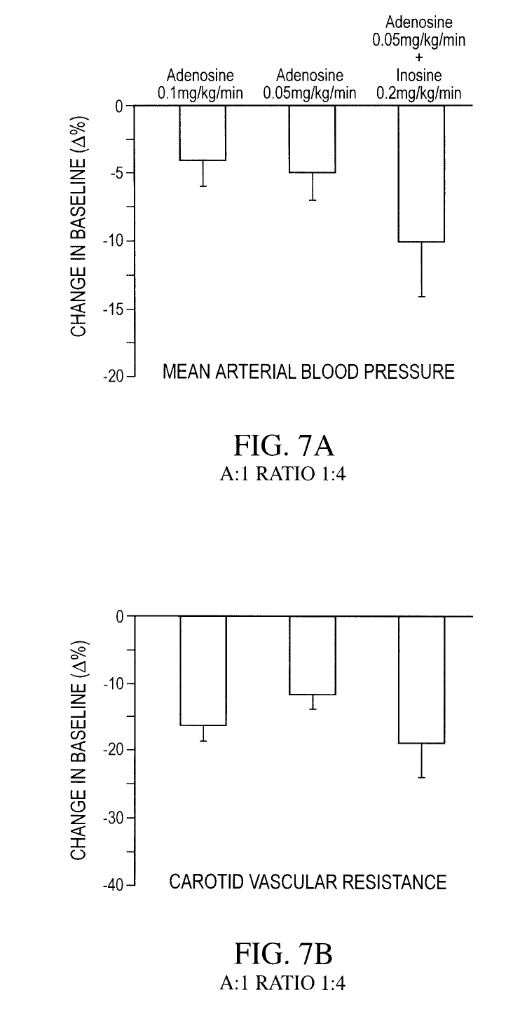

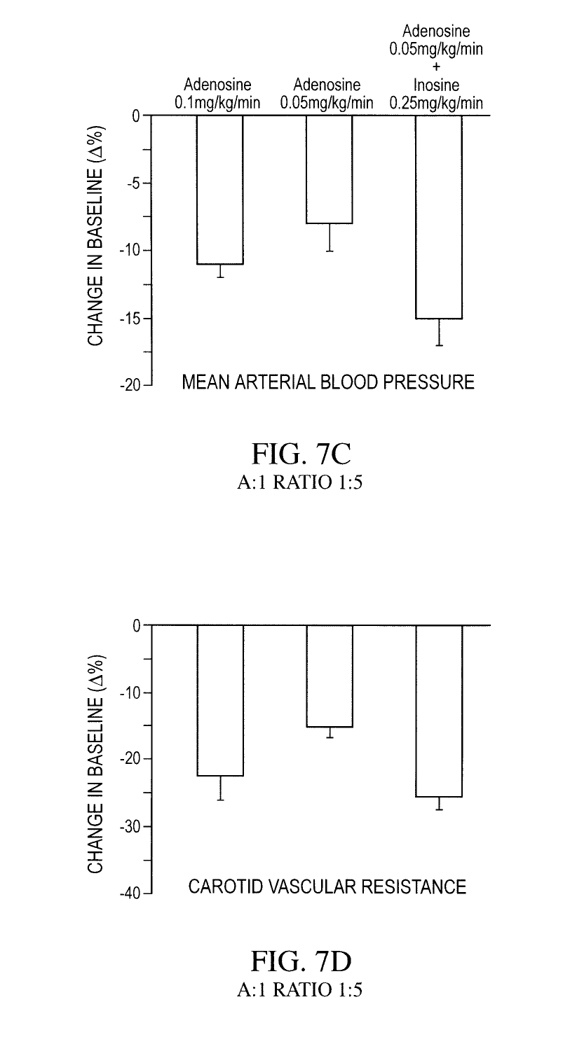

In another series of experiments, the effects of adenosine and adenosine:inosine combination (BIDOSINE) on mean arterial blood pressure and carotid vascular resistance were studied. Results are presented in FIGS. 6A-6F and 7A-7D.

FIGS. 6A-6F depict the effects of the combination of adenosine and inosine on mean arterial blood pressure and carotid vascular resistance. With adenosine fixed at half adenosine max, as A:I ratios increase from 1:1 to 1:3, the effect of the combination on (decreasing) arterial resistance and (decreasing) blood pressure progressively increases, equaling the effects of adenosine max on mean at an A:I ratio of 1:3. Panels 6A and 6B show effects, respectively, on mean arterial blood pressure and carotid vascular resistance of continuous infusions of adenosine alone (0.1 mg/kg/min, adenosine max), adenosine alone (0.05 mg/kg/min, half adenosine max), and adenosine at half adenosine max+inosine (0.05 mg/kg/min, for an A:I ratio of 1:1). Panels 6C and 6D respectively show effects on mean arterial pressure and carotid vascular resistance of continuous infusions of adenosine alone (0.1 mg/kg/min, adenosine max), adenosine alone (0.05 mg/kg/min, half adenosine max), and adenosine at half adenosine max+inosine (0.1 mg/kg/min, for an A:I ratio of 1:2). Panels 6E and 6F show effects on mean arterial pressure and carotid vascular resistance of continuous infusions of adenosine alone (0.1 mg/kg/min, adenosine max), adenosine alone (0.05 mg/kg/min, half adenosine max), and adenosine at half adenosine max+inosine (0.15 mg/kg/min, for an A:I ratio of 1:3).

FIGS. 7A-7D further illustrate that, with adenosine at half adenosine max, at A:I ratios of 1:4 and above, effects on (decreasing) blood pressure and (decreasing) arterial resistance exceed that of adenosine alone at its maximally effective dosage. Together with the data in FIGS. 6A-6F, FIGS. 7A-7D demonstrate dose-related effects on blood pressure as the adenosine:inosine ratio is increased from 1:1 to 1:5.

The fall in blood pressure observed in these studies is mainly due to the concomitant decrease of vascular resistance. Heart rate was slightly reduced, however, at various A:I combination ratios, which may also contribute to reduced pressure. Although a statistically insignificant trend towards heart rate reduction with increasing inosine concentrations was observed in these studies (see Table 1, below), it should be noted that anesthesia is known to suppress sympathetic stimulation of the heart. It would appear unlikely, therefore, that a similar heart rate trend would be observed in alert (that is, awake), animals, including patients.

TABLE 1

| Adenosine:Inosine Ratio | Heart Rate Modification (%) |
|---|---|
| 1:1 | Stable, with no change |
| 1:2 | −6 ± 2 |
| 1:3 | −6 ± 1 |
| 1:4 | Stable, with no change |
| 1:5 | −7 ± 2 |

In summary, these experiments demonstrate that inosine, administered concurrently with adenosine by continuous intravenous infusion, is capable of potentiating certain of adenosine's physiological effects, permitting adenosine to be administered at half the dose required as a single agent, to achieve maximal increase in carotid arterial blood flow. At certain weight ratios, inosine acts synergistically with adenosine. It can also be concluded that (i) inosine alone is poorly effective; ((ii) the combination of adenosine and inosine can modulate arterial resistance.

### Example 2

### Inosine Combined With Reduced Dose Adenosine Improves Coronary Flow and Cardiac Output With Fewer Side Effects In Sheep

An experimental sheep model, developed for invasive hemodynamic measurements under various conditions, was used to study the potentiating and synergistic effects of inosine on continuous adenosine infusion. The model is described in Tanoue et al., “Ischemic preconditioning reduces unloaded myocardial oxygen consumption in an in-vivo sheep model,” Cardiovascular Research 55: 633-641 (2002) and Xia et al., “Remote preconditioning lessens the deterioration of pulmonary function after repeated coronary artery occlusion and reperfusion,” Canadian Journal of Anesthesia 50: 481-488 (2003), the disclosures of which are incorporated herein by reference in their entireties. A total of twelve animals were studied.

Briefly, the sheep were premedicated, anesthetized, intubated, and ventilated with positive pressure ventilation. Electrocardiogram (ECG) was continuously followed. The carotid artery was surgically denuded to place an arterial line for arterial blood pressure (ABP) measurement. Denuding of the jugular vein was performed to place a double or triple lumen central venous line for measurement of central venous pressure (CVP) and fluid and drug administration. A small left thoracotomy was made in the third or fourth intercostal space and the heart suspended in a pericardial cradle. A flow probe (transonic probe) was placed around the pulmonary artery and around the left circumflex coronary artery. A catheter was inserted in the pulmonary artery to determine pulmonary arterial pressure. In addition, 9 animals received a conductance catheter which was positioned in the left and right ventricle and preload-afterload variations were made by occlusion and/or constriction of the caval veins and/or pulmonary artery and/or aorta in order to study myocardial contractility. Regional blood flow measurements were also performed using a colored microsphere injection technique.

Each animal served as its own control. The sequence of measurements was always the same: 1) under baseline conditions; 2) under adenosine; 3) 15 minutes stabilization, then new measurements under baseline conditions; 4) measurements under bidosine. From the first three animal experiments, it was determined that adenosine 200 μg/kg/min was the optimal single agent adenosine dose, i.e., the dose ensuring the highest coronary flow (adenosine max). Therefore, all further experiments compared this dose to the BIDOSINE formulation containing 100 μg/kg/min (that is, half adenosine max) adenosine+500 μg/kg/min inosine (A:I ratio of 1:5). Results are given in Table 2.

TABLE 2
Cardiac hemodynamic data in the sheep

| | | | |
|---|---|---|---|
| 1) Baseline | 2) Adenosine | 3) Baseline | 4) BIDOSINE |

Heart rate (HR)

| | | | | |
|---|---|---|---|---|
| mean | 89.0375 | 90.6375 | 89.825 | 93.3125 |
| SEM | 5.104373 | 5.531692 | 5.564195 | 5.803399 |

Systolic left ventricular pressure (LVP_{sys})

| | | | | |
|---|---|---|---|---|
| mean | 77.8625 | 59.35 | 78.9375 | 67.55 |
| SEM | 3.498721 | 3.116431 | 5.655937 | 6.980253 |

Left ventricular dP/dtₘₐₓ (contractility) (dpₘₐₓ)

| | | | | |
|---|---|---|---|---|
| mean | 1110 | 1077.714 | 1295.875 | 1475.125 |
| SEM | 70.96377 | 63.01765 | 152.2697 | 333.8587 |

Left ventricular dP/dtₘᵢₙ (relaxation) (dpₘᵢₙ)

| | | | | |
|---|---|---|---|---|
| mean | −1268.86 | −821.286 | −1247 | −890 |
| SEM | 100.8682 | 58.60348 | 143.0898 | 132.0914 |

Central venous pressure (CVP)

| | | | | |
|---|---|---|---|---|
| mean | 9.325 | 9.4875 | 8.9875 | 9.3 |
| SEM | 1.216956 | 1.131282 | 1.142903 | 1.12726 |

Mean arterial blood pressure (ABPₘ)

| | | | | |
|---|---|---|---|---|
| mean | 66.35 | 43.2625 | 65.9 | 49.0875 |
| SEM | 3.690286 | 2.648985 | 5.714018 | 5.766821 |

Mean pulmonary arterial pressure (pupₘ)

| | | | | |
|---|---|---|---|---|
| mean | 16.5625 | 16.425 | 17.25 | 17.75 |
| SEM | 1.201181 | 1.150272 | 1.192237 | 1.209634 |

Cardiac output (co)

| | | | | |
|---|---|---|---|---|
| mean | 4179.5 | 4943 | 4380.625 | 5072.875 |
| SEM | 385.1477 | 448.6707 | 337.1507 | 427.7918 |

Coronary flow (cf)

| | | | | |
|---|---|---|---|---|
| mean | 50.775 | 129.55 | 57.875 | 145.5375 |
| SEM | 11.93787 | 27.62579 | 12.94075 | 33.16207 |

BIDOSINE showed at least equal efficacy in terms of coronary flow and cardiac output, with fewer side effects.

In particular, coronary blood flow was observed to go from 50.7 cms/sec at the initial base line to 129.5 cms/sec with adenosine administered as a single agent, and from 57.8 at the second baseline, to 145.5 cms/sec, with continuous i.v. infusion of BIDOSINE.

Coronary output with BIDOSINE (5072.9) was as high as with adenosine as a single agent (4943), and above baseline.

Systolic, diastolic and mean arterial blood pressure decreased significantly more with adenosine than with BIDOSINE.

Left ventricular systolic pressure decreased significantly more with adenosine than with BIDOSINE.

Central venous pressure showed no difference.

Heart rate increased slightly, but significantly more with BIDOSINE.

dP/dtₘₐₓ showed no difference.

dP/dtₘᵢₙ worsened significantly more with adenosine than with bidosine.

No differences were noted regarding diastolic and mean pulmonary artery pressure.

Regional blood flows, as well as potential changes in the distribution of coronary flow into different parts of the myocardium, were also studied. This was done by using a colored microsphere technique, essentially as described in Muller et al., “Developmental changes in cerebral autoregulatory capacity in the fetal sheep parietal cortex,” J Physiol. 539(Pt 3):957-67 (2002); Miller et al., “Physiological evidence for arteriovenous anastomoses in the uterine circulation of late-pregnant ewes,” Clin. Exp. Pharmacol. Physiol. 25(2):92-8 (1998); Herijgers et al., “Changes in organ perfusion after brain death in the rat and its relation to circulating catecholamines,” Transplantation 62: 330-335 (1996); and Wouters et al., “Ischemic event characteristics determine the extent of myocardial stunning in conscious dogs,” Basic Res. Cardiol. 91(2):140-6 (1996), the disclosures of which are incorporated herein by reference in their entireties.

Briefly, colored microspheres were injected at baseline, and after 5 minutes of adenosine or BIDOSINE administration. At necropsy, tissue was digested and the number of colored microspheres per gram of wet weight in a tissue sample determined. By comparing these numbers with the number of microspheres in a reference sample drawn from the aorta at a predetermined rate, we could quantify the absolute flow rate to the tissue sample at the moment when the microspheres were injected. The statistical analysis was performed by the same methodology as for the hemodynamic parameters.

The results of the microsphere experiments are presented in Table 3. Values are expressed in ml/min/g tissue (wet weight). LV=left ventricle; RV=right ventricle; endo=endocardial half; epi=epicardial half. As in Table 2, the numbers reflect the sequence of experimental conditions, as follows: 1) under baseline conditions; 2) under adenosine; 3) 15 minutes stabilization, then new measurements under baseline conditions; 4) measurements under bidosine.

TABLE 3
Regional blood flows
Left ventricle

| experiment | Lvendo1 | Lvendo2 | Lvendo3 | Lvendo4 | Lvepi1 | Lvepi2 | Lvepi3 | Lvepi4 |
|---|---|---|---|---|---|---|---|---|
| Herado4 | 0.928 | 2.04 | 0.894 | 1.392 | 0.939 | 2.829 | 0.764 | 2.129 |
| Herado5 | 0.7 | 0.873 | 0.973 | 1.695 | 0.847 | 1.558 | 1.109 | 2.75 |
| Herado6 | 1.027 | 3.119 | 1.252 | 1.968 | 1.173 | 3.441 | 1.244 | 1.989 |
| Herado8 | 1.209 | 1.662 | 1.342 | 1.429 | 0.267 | 0.603 | 0.256 | 0.583 |
| Herado9 | 0.59 | 0.854 | 0.581 | 0.335 | 0.778 | 0.903 | 0.913 | 1.205 |
| Herado7 | 0.636 | 1.704 | 1.254 | 4.635 | 0.712 | 1.386 | 1.137 | 3.204 |
| Herado10 | 0.695 | 0.167 | 0.736 | 3.78 | 0.837 | 0.629 | 1.045 | 5.337 |
| Herado11 | 0.684 | 3.6 | 0.555 | 3.448 | 0.602 | 3.028 | 0.669 | 2.952 |
| Herado12 | 0.846 | 1.854 | 0.761 | 1.749 | 0.914 | 3.977 | 1.154 | 2.14 |
| mean | 0.812778 | 1.763667 | 0.927556 | 2.270111 | 0.785444 | 2.039333 | 0.921222 | 2.476556 |
| SD | 0.194741 | 1.02619 | 0.280895 | 1.298575 | 0.236793 | 1.21665 | 0.295604 | 1.277424 |

Right Ventricle

| | RVendo1 | RVendo2 | RVendo3 | RVendo4 | RVepi1 | RVepi2 | RVepi3 | RVepi4 |
|---|---|---|---|---|---|---|---|---|
| Herado4 | 0.748 | 3.164 | 0.651 | 2.111 | 0.621 | 2.118 | 0.785 | 1.774 |
| Herado5 | 0.752 | 1.831 | 1.142 | 3.183 | 0.55 | 1.08 | 0.83 | 1.952 |
| Herado6 | 1.116 | 3.944 | 1.284 | 2.374 | 0.947 | 2.795 | 1.061 | 1.967 |
| Herado8 | 0.987 | 2.286 | 1.052 | 2.117 | 1.027 | 1.455 | 1.085 | 1.374 |
| Herado9 | 0.83 | 1.146 | 0.913 | 1.501 | 0.947 | 1.354 | 1.796 | 2.527 |
| Herado7 | 0.588 | 0.922 | 0.731 | 2.427 | 1.329 | 0.957 | 1.075 | 2.757 |
| Herado10 | 0.655 | 0.235 | 0.801 | 4.342 | 0.983 | 1.119 | 1.333 | 4.595 |
| Herado11 | 0.522 | 3.74 | 0.604 | 3.226 | 0.662 | 2.647 | 1.296 | 2.402 |
| Herado12 | 0.713 | 2.776 | 1.072 | 1.759 | 0.727 | 2.027 | 0.919 | 1.704 |
| mean | 0.767889 | 2.227111 | 0.916667 | 2.56 | 0.865889 | 1.728 | 1.131111 | 2.339111 |
| SD | 0.187841 | 1.295018 | 0.235679 | 0.88179 | 0.246961 | 0.691392 | 0.311381 | 0.95167 |

In the left endocardium, adenosine increases vasodilation, with regional blood flow going from 0.812 ml/min/g at base line to 1.763. BIDOSINE increases flow from 0.927 ml/min/g at baseline to 2.270. Similar increases are observed in the left epicardium. Epicardial vasodilatation is more pronounced than endocardial vasodilatation in the left ventricle, but not in the right ventricle.

Overall, adenosine and BIDOSINE 1:5 have the same effects on coronary vasodilatation: both improve cardiac haemodynamics. However, BIDOSINE provides these effects with fewer side effects, notably a much reduced drop in blood pressure.

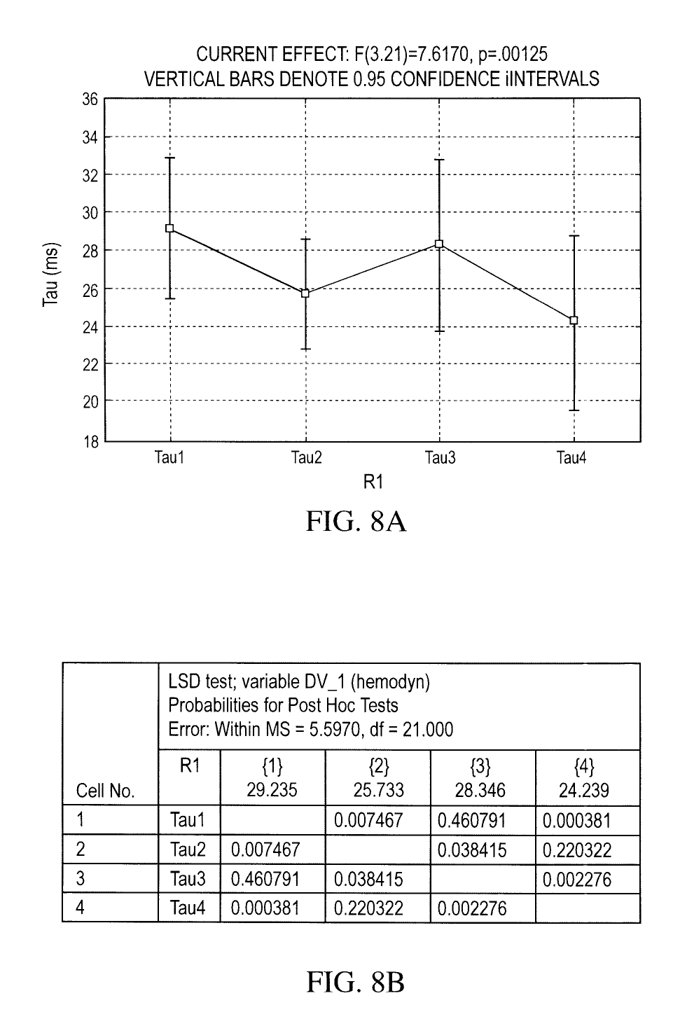

In addition, both adenosine as single agent, and BIDOSINE, advantageously affect left ventricular relaxation. FIGS. 8A and 8B show the effects of adenosine and BIDOSINE in this experimental model on left ventricular relaxation constant (Tau). Tau (left ventricular relaxation constant) is a parameter for relaxation. Tau is less load-dependent than dP/dtₘᵢₙ. The data demonstrate that adenosine at adenosine max and BIDOSINE (adenosine at half adenosine max, A:I ratio of 1:5) both increase ejection fraction and stimulate systolic function, and also improve diastolic function. The data further show that BIDOSINE performs slightly better than adenosine. Specifically, Tau is significantly shorter after adenosine and BIDOSINE compared with baseline (ANOVA p=0.001). The change in Tau by adenosine and BIDOSINE is comparable. This means that both products improve relaxation. This result appears contrary to the effect described by dP/dtₘᵢₙ (see Table 2), the discrepancy caused by the load-dependency of dP/dtₘᵢₙ, with BIDOSINE having less effect on loading conditions than adenosine.

### Example 3

### BIDOSINE Prevents Reperfusion Injury After Myocardial Ischemia in the Rat

The purpose of the study was to evaluate the effects of adenosine and a combination of adenosine (at half adenosine max) with inosine (at a 1:5 A:I weight ratio) (BIDOSINE 1:5), in reducing or prevention reperfusion injury. In these experiments, the effects of continuous infusion of adenosine and of BIDOSINE 1:5 on the left ventricular ejection fraction (EF) were assessed after creation of left myocardial ischemia in the rat. The key objective was to verify that BIDOSINE could reduce reperfusion injury with equal or greater efficacy than adenosine administered as a single agent at its maximally effective intravenous dose.

A total of 24 male Wistar rats were anesthetized with a mixture of ketamine (Ketalar) 50 mg/kg, and acepromazine (Vetranquil) 2 mg/kg, using intramuscular injections. After oral intubation, rats were mechanically ventilated with air (Harvard Rodent Ventilator model 683) and fixed with their left side up. A left thoracotomy was performed via the third intercostal space, and muscles and pericardium was carefully dissected. The LAD (left anterior descending) coronary artery was localized using magnifying glasses, and ligated with a 6-0 non-absorbable Prolene suture just proximal to the bifurcation of the LAD.

The coronary suture was released after 35 minutes of occlusion. During the following 60 minutes, agents were administered continuously through the femoral vein, using an electrical infusion pump. Rats were given either (i) adenosine at 0.1 mg/kg/min (adenosine max), (ii) adenosine at 0.05 mg/kg/min (half adenosine max) plus inosine at 0.25 mg/kg/min, for an A:I weight ratio of 1:5; or saline. The intercostal space was closed with a 6-0 non-absorbable Prolene suture and the skin with a 5-0 absorbable Vicryl suture.

Echocardiographic studies were performed at postoperative day 2 (baseline) and at postoperative day 30.

TABLE 4 shows end-diastolic volume (EDV), end-systolic volume (ESV), and ejection fraction (EF) at postoperative day 2 (baseline) and at postoperative day 30 for the adenosine, BIDOSINE, and control (saline) groups.

TABLE 4
Effects on left ventricular ejection fraction
BIDOSINE 1:5 GROUP

| Post-operative day 2 | | | Post-operative day 30 | | |
|---|---|---|---|---|---|
| EDV | ESV | EF | EDV | ESV | EF |
| 0.14 | 0.05 | 0.64 | 0.28 | 0.12 | 0.59 |
| 0.45 | 0.24 | 0.47 | 0.44 | 0.13 | 0.71 |
| 0.20 | 0.05 | 0.73 | 0.25 | 0.08 | 0.67 |
| 0.27 | 0.06 | 0.79 | 0.27 | 0.11 | 0.58 |
| 0.33 | 0.12 | 0.63 | 0.36 | 0.19 | 0.48 |
| 0.33 | 0.13 | 0.61 | 0.29 | 0.10 | 0.66 |
| 0.46 | 0.18 | 0.61 | 0.46 | 0.18 | 0.61 |
| 0.42 | 0.22 | 0.47 | 0.42 | 0.22 | 0.47 |
| 0.32 | 0.13 | 0.62 | 0.35 | 0.14 | 0.60 |

ADENOSINE GROUP

| Post-operative day 2 | | | Post-operative day 30 | | |
|---|---|---|---|---|---|
| EDV 1 | ESV | EF | EDV | ESV | EF |
| 0.41 | 0.14 | 0.67 | 0.44 | 0.17 | 0.61 |
| 0.35 | 0.13 | 0.64 | 0.42 | 0.29 | 0.32 |
| 0.37 | 0.15 | 0.60 | 0.33 | 0.12 | 0.65 |
| 0.48 | 0.24 | 0.49 | 0.36 | 0.20 | 0.44 |
| 0.40 | 0.16 | 0.60 | 0.38 | 0.20 | 0.59 |

CONTROL GROUP

| Post-operative day 2 | | | Post-operative day 30 | | |
|---|---|---|---|---|---|
| EDV | ESV | EF | EDV | ESV | EF |
| 0.40 | 0.23 | 0.42 | 0.50 | 0.25 | 0.50 |
| 0.47 | 0.23 | 0.51 | 0.43 | 0.29 | 0.31 |
| 0.34 | 0.16 | 0.55 | 0.05 | 0.03 | 0.25 |
| 0.42 | 0.10 | 0.77 | 0.33 | 0.10 | 0.70 |
| 0.45 | 0.22 | 0.53 | 0.39 | 0.25 | 0.36 |
| 0.33 | 0.10 | 0.71 | 0.39 | 0.15 | 0.62 |
| 0.40 | 0.17 | 0.58 | 0.34 | 0.18 | 0.46 |

The data demonstrate that continuous intravenous infusion of adenosine preserved ejection fraction when administered immediately after ischemic insult at its maximally effective dose (ejection fraction 59%). The data further demonstrate that continuous infusion of a combination of adenosine at half its maximally effective dose with inosine at a 1:5 A:I weight ratio provide comparable protection (ejection fraction 60%). The control group ejection fraction at day 30 was only 46%.

The experiment was repeated with 45 minute period of ischemia, and with the addition of a further BIDOSINE experimental group. The BIDOSINE composition for this additional group comprised adenosine, administered at 0.01 mg/kg/min ( 1/10 its single agent maximally effective dose) with inosine 0.20 mg/kg/min, for an A:I weight ratio of 1:20.

Preliminary results (n=5 per group) of this ongoing study are given in Table 5, below, reporting day 30 post ischemia results. Although statistical significance has not yet been reached, there is surprisingly a clear trend in favor of BIDOSINE 1:20 over adenosine alone and BIDOSINE 1:5 (adenosine 0.05 mg/kg/min plus inosine at 0.25 mg/kg/min) with left ventricular EF (LVEF) respectively at 69%, 45% and 34%.

TABLE 5

| GROUP | EDV | ESV | LVEF |
|---|---|---|---|
| BIDOSINE 1:20 | 0.49 | 0.15 | 0.69 |
| BIDOSINE 1:5 | 0.58 | 0.39 | 0.34 |
| ADENOSINE | 0.47 | 0.26 | 0.45 |

### Example 4

### BIDOSINE Effects on Spinal Cord Injury In Rats

Previous studies of the microvasculature after acute spinal cord injury (SCI) have demonstrated that one of the main contributors to long-term disability is a secondary disorder of the microcirculation.

The objective of the present study was to evaluate the potential neuroprotective effects of 30 minute and 60 minute continuous intravenous (i.v.) infusion of BIDOSINE 1:5, a combination of adenosine at 0.05 mg/kg/min (half adenosine max) with inosine at 0.25 mg/kg/min, for an A:I weight ratio of 1:5. BIDOSINE administration was compared to adenosine (0.1 mg/kg/min), administered by continuous i.v. infusion from thirty to sixty minutes after the injury (n=13 in each group). As controls, a group of animals (n=11) received a 60-min IV perfusion of saline solution starting 30 min after the injury. The potential neuroprotective effect of the compound was evaluated by performing various behavioral studies.

In these experiments, the photothrombotic model of ischemia was chosen over the occlusion model (aortic clamp), since it better reproduces the pathophysiology of an ischemic insult. The photothrombotic model has the advantages of (i) causing a permanent focal ischemia with reperfusion processes that more perfectly mimics the pathophysiological conditions observed in clinics, and (ii) affecting a controlled volume of nervous tissue. Moreover, the location of the lesion can be restricted to precise regions (three to four metamers). The injury is created using a beam of a xenon lamp conveyed by fiberoptics on the selected vertebral site. The irradiation is performed over the translucent dorsal surface of the vertebral laminae. The green light (560 nm) induces the excitation of the previously injected dye (Rose Bengal) present in the spinal cord microvasculature. The resultant photochemical reaction releases non-radical reactive oxygen species which damage the endothelium of medullary vessels and induce platelet aggregation and thrombosis in the microvasculature.

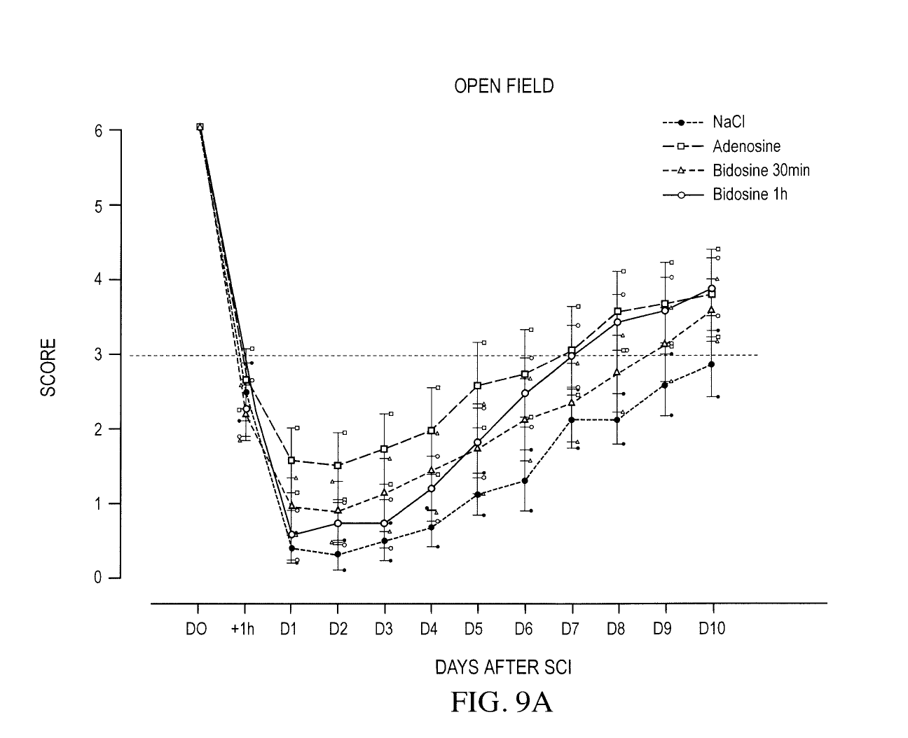

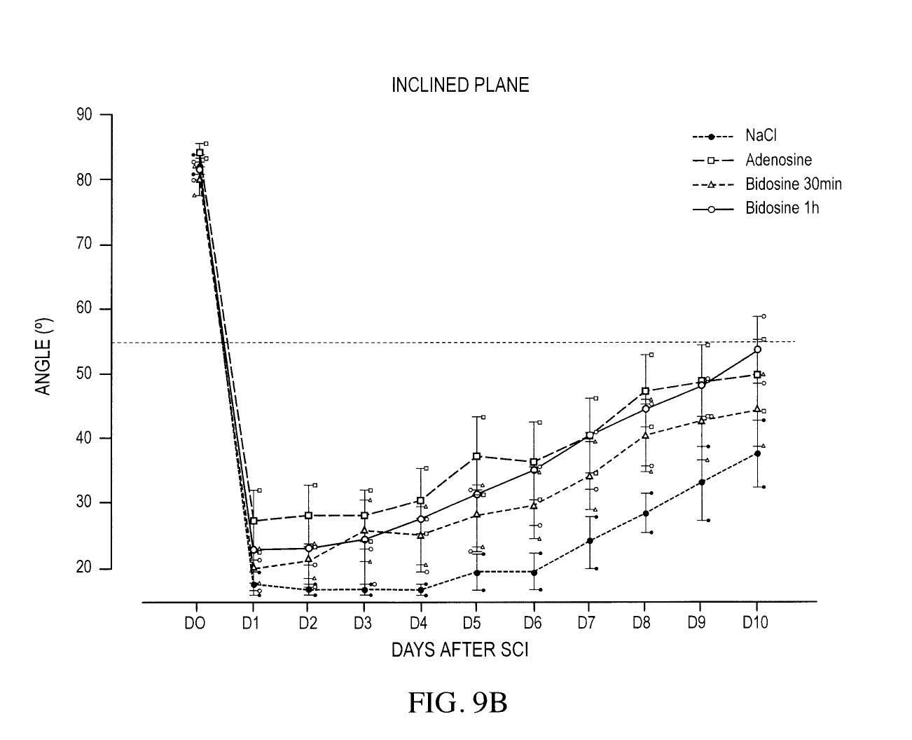

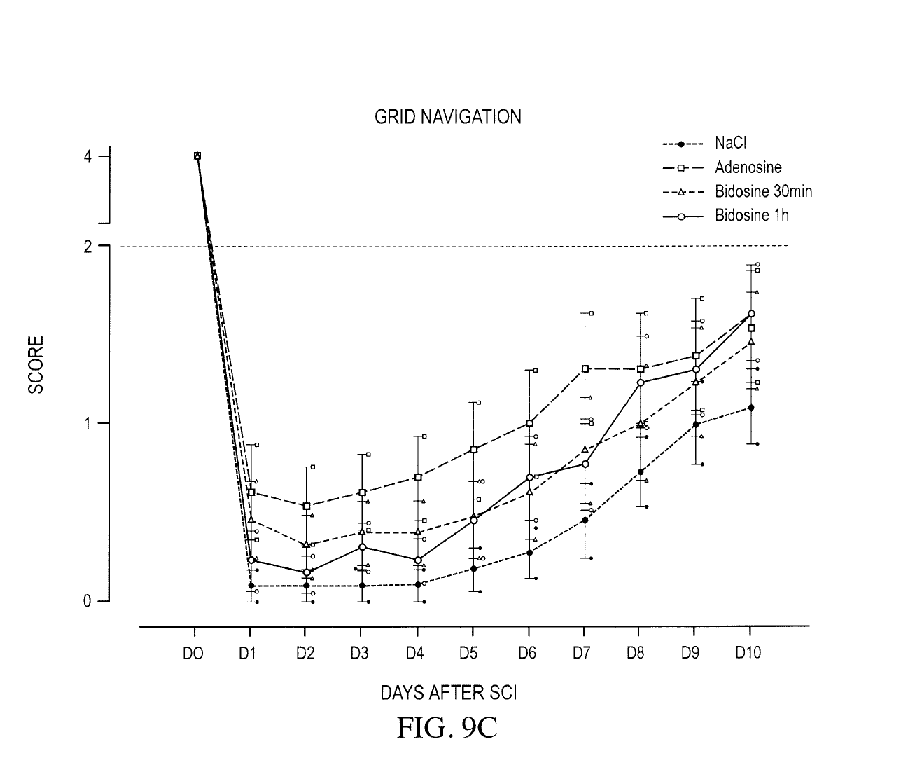

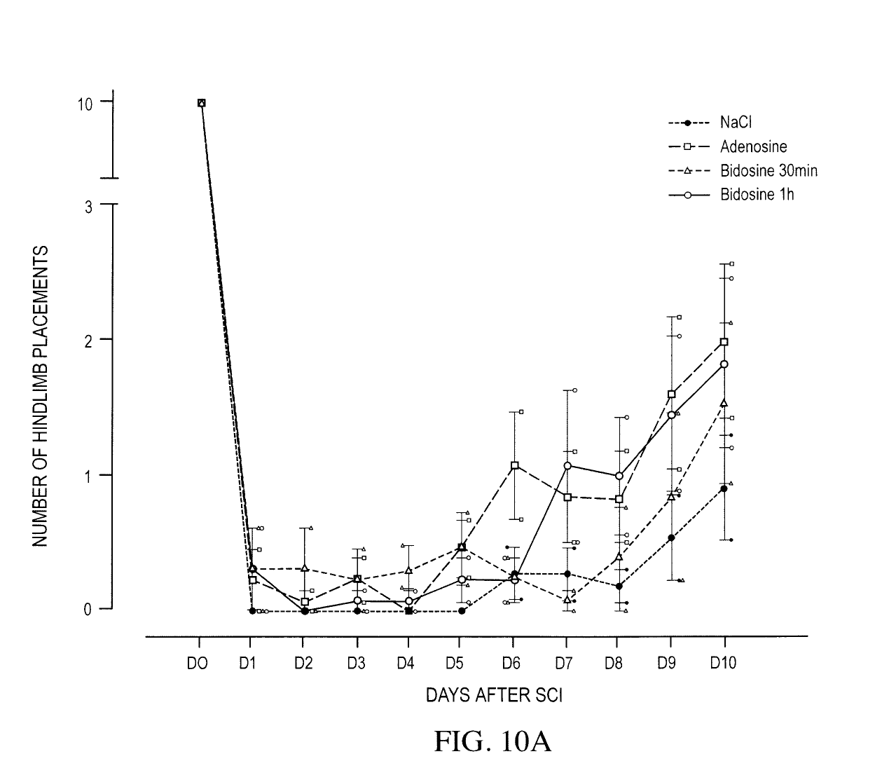

Results are summarized in FIGS. 9-11, which demonstrate that BIDOSINE 1:5 equals adenosine at day 10 post-injury (D10) for all tested functions, and does better than adenosine for recovery of bladder function. In addition, the rate of recovery from days D4 to D10 is faster with BIDOSINE. BIDOSINE hastens the behavioral recovery in a rat model of spinal cord ischemia, with results equivalent on multiple tests to adenosine effects at 0.1 mg/kg/min. Results are shown to day 10 post-injury.

FIGS. 9A-9C show results of an open field test (FIG. 9A), inclined plane test (FIG. 9B), and Grid navigation (FIG. 9C). Statistically significant improvements over control are demonstrated in gross and fine motor functional outcomes, compared to controls (n=12/group) (BIDOSINE vs. saline, two way ANOVA p<0.001), with both adenosine and BIDOSINE.

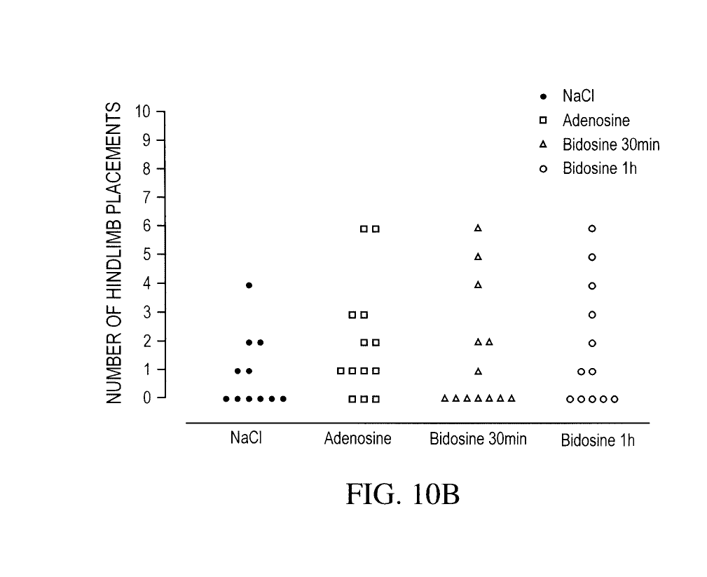

FIGS. 10A-10B show that BIDOSINE 1:5 (adenosine at 0.05 mg/kg/min, half adenosine max, with inosine at 0.25 mg/kg/min), hastens the return of proprioception, with results equivalent to adenosine effects at 0.1 mg/kg/min.

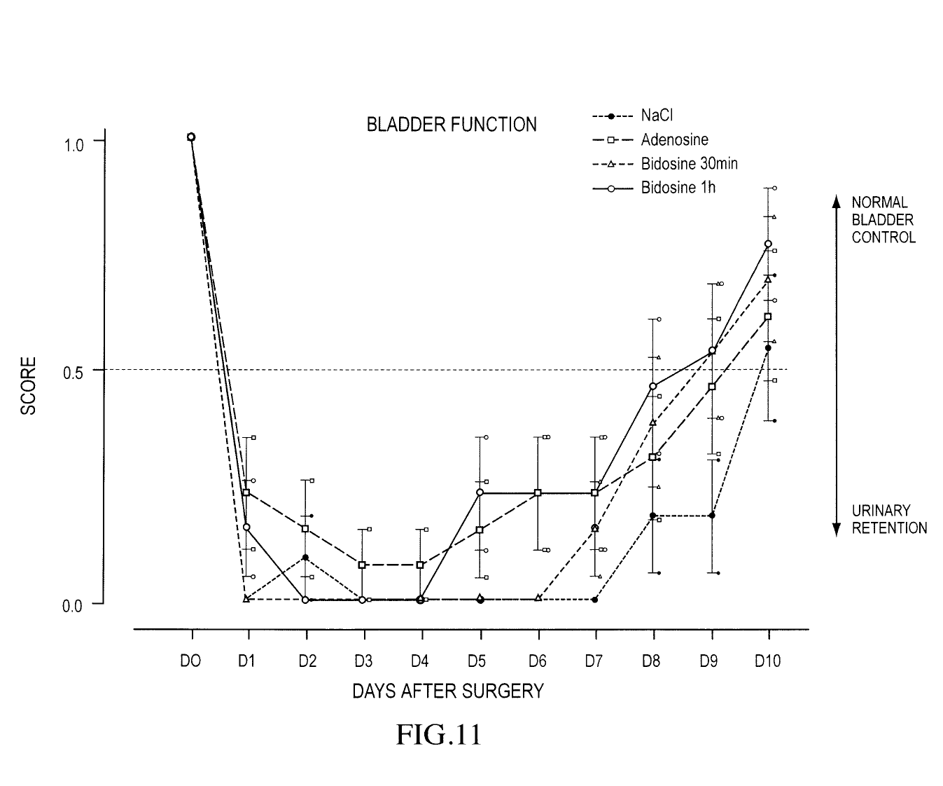

FIG. 11 shows data comparing bladder function (BIDOSINE vs. saline, two way ANOVA p<0.001), demonstrating that BIDOSINE hastens return of bladder function after spinal cord injury.

The data demonstrate that continuous i.v. infusion of adenosine alone at adenosine max dosage provided best efficacy until day 4. However, the behavioral follow-up consistently revealed a faster improvement of the motor, sensory and reflex scores in the 60 minute BIDOSINE group compared to the others. Thus, at day 10 (D10), animals in this group reached scores that were very comparable to those obtained with the adenosine treatment. This time course of recovery suggests that treatment with BIDOSINE for 60 minutes after the insult may improve the functionality of the spared tissue, and leads to faster rates of behavioral recovery (in particular for the bladder function). Additionally, analysis of the motor and the sensory score curves suggests that at longer time follow-up times, treatment with BIDOSINE for 60 minutes after the injury might be more beneficial than the early treatment with adenosine.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A pharmaceutical composition comprising a combination of pharmaceutically active ingredients, the combination consisting essentially of adenosine and inosine in an adenosine:inosine weight ratio of about 1:1 to about 1:9.

2. A pharmaceutical composition comprising a combination of pharmaceutically active ingredients, the combination consisting essentially of adenosine and inosine in an adenosine:inosine weight ratio of about 1:1 to about 20:1.

3. (canceled)

4. The pharmaceutical composition according to claim 1, in which the ratio is about 1:3 to about 1:6.

5. The pharmaceutical composition according to claim 4, in which the ratio is about 1:4.

6. The pharmaceutical composition according to claim 4, in which the ratio is about 1:5.

7. The pharmaceutical composition according to claim 2, in which the ratio is about 10:1.

8. The pharmaceutical composition according to claim 2, in which the ratio about is 7:1 to about 4:1.

9. The pharmaceutical composition according to claim 1, in which the pharmaceutical composition is suitable for intravenous, intra-atrial, or intra-arterial infusion.

10. (canceled)

11. (canceled)

12. (canceled)

13. The pharmaceutical composition according to claim 9, wherein adenosine and inosine are present at concentrations suitable for intravenous administration at an adenosine dosage rate of 50-70 μg/kg/min and an inosine dosage rate of 10-35 μg/kg/min.

14. (canceled)

15. The pharmaceutical composition according to claim 1, wherein the concentration of adenosine is about 0.5 to about 4 mg/ml.

16. (canceled)

17. (canceled)

18. The pharmaceutical composition according to claim 15, wherein the concentration of adenosine is about 3 mg/ml.

19. The pharmaceutical composition according to claim 15, wherein the concentration of adenosine is about 4 mg/ml.

20. The pharmaceutical composition according to claim 15, wherein the concentration of inosine is about 0.3 to about 20 mg/ml.

21. (canceled)

22. The pharmaceutical composition according to claim 20, wherein the concentration of inosine is about 3 to about 4 mg/ml.

23. (canceled)

24. (canceled)

25. The pharmaceutical composition according to claim 20, wherein the concentration of inosine is about 10 mg/ml.

26. (canceled)

27. The pharmaceutical composition according to claim 20, wherein the concentration of inosine is about 15 mg/ml.

28. The pharmaceutical composition according to claim 20, wherein the concentration of inosine is about 18 to about 20 mg/ml.

29. A unit dosage form containing about 7-30 ml of a pharmaceutical composition comprising adenosine and inosine in an adenosine:inosine weight ratio of about 1:1 to about 1:9, wherein the pharmaceutical composition is a sterile, nonpyrogenic, fluid.

30. The unit dosage form of claim 29 containing about 5 ml.

31. The unit dosage form of claim 29 containing about 10 ml.

32. The unit dosage form of claim 29 containing about 15 ml.

33. A unit dosage form containing about 200-750 ml of a pharmaceutical composition comprising adenosine and inosine in an adenosine:inosine weight ratio of about 1:1 to about 1:9, wherein the pharmaceutical composition is a sterile, nonpyrogenic, fluid.

34. (canceled)

35. (canceled)

36. In a method of pharmacologic stress testing, the improvement comprising:
concurrently administering adenosine and inosine to induce the pharmacologic stress, wherein adenosine and inosine are administered in an adenosine:inosine ratio of about 1:1 to about 1:20.

37. The method of claim 36, wherein adenosine and inosine are administered by intravenous infusion.

38. The method of claim 37, wherein adenosine and inosine are administered by intravenous infusion of a composition comprising adenosine and inosine in an adenosine:inosine ratio of about 1:1 to about 1:20.

39. The method of claim 38, wherein the adenosine:inosine ratio is about 1:4.

40. The method of claim 38, wherein the adenosine:inosine ratio is about 1:5.

41. The method of claim 36, wherein adenosine is infused at less than about 140 μg/kg/min.

42. The method of claim 41, wherein adenosine is infused at no more than about 70 μg/kg/min.

43. The method of claim 36, wherein adenosine and inosine are administered continuously for a period of at least 2 minutes.

44. The method of claim 43, wherein adenosine and inosine are administered continuously for a period of greater than 2 minutes but less than 6 minutes.

45. A method of pharmacologic stress testing, the method comprising:
concurrently administering adenosine and inosine to induce pharmacologic stress, wherein adenosine and inosine are administered in an adenosine:inosine ratio of about 1:1 to about 1:20; and
assessing one or more parameters of cardiac function during or after the infusion.

46. The method of claim 36 or 45, wherein assessing cardiac function includes use of one or more techniques selected from the group consisting of: electrocardiography, M mode echography, two dimensional echography, three dimensional echography, echo-doppler, cardiac imaging, planar (conventional) scintigraphy, single photon emission computed tomography (SPECT), dynamic single photon emission computed tomography, positron emission tomography (PET), first pass radionuclide angiography, equilibrium radionuclide angiography, nuclear magnetic resonance (NMR) imaging, perfusion contrast echocardiography, digital subtraction angiography (DSA), and ultrafast x-ray computed tomography (CINE CT).

47. The method of claim 45, wherein assessing cardiac function is performed by SPECT.

48. The method of claim 45, wherein assessing cardiac function is performed by PET.

49. A method of treating post-ischemic myocardial injury, the method comprising:
administering at least a first concurrent parenteral infusion of adenosine and inosine during or following an acute cardiac ischemic event, wherein the adenosine and inosine are infused at an A:I ratio of about 1:1 to about 1:20 or infused at an A:I ratio of about 1:1 to 20:1.

50. The method of claim 49, wherein the acute ischemic event is a myocardial infarction.

51. The method of claim 49, wherein adenosine and inosine are infused in a pharmaceutical composition comprising adenosine and inosine in an adenosine:inosine weight ratio of about 1:1 to about 1:20.

52. The method of claim 49, wherein adenosine and inosine are infused intravenously.

53. The method of claim 52, wherein adenosine is infused at a rate of less than about 140 μg/kg/min.

54. The method of claim 53, wherein adenosine is infused at a rate of less than about 80 μg/kg/min.

55. The method of claim 54, wherein adenosine is infused at a rate of about 35-70 μg/kg/min.

56. (canceled)

57. The method of claim 52, wherein inosine is infused at a rate of 35-210 μg/kg/min.

58. The method of claim 52, wherein inosine is infused at a rate of 200-600 μg/kg/min.

59. The method of claim 52, wherein inosine is infused at a rate of 10-30 μg/kg/min.

60. The method of claim 49, wherein the first parenteral infusion is begun within 6 hours of onset of acute ischemia.

61. The method of claim 49, further comprising at least a second concurrent parenteral infusion of adenosine and inosine, wherein adenosine and inosine are infused at an A:I ratio of about 1:1 to about 1:20 or infused at an A:I ratio of about 1:1 to about 20:1.

62. The method of claim 49, wherein each infusion is of at least 30 minutes duration.

63. A method of treating acute injury to the central or peripheral nervous system, the method comprising:
administering at least a first concurrent parenteral infusion of adenosine and inosine during or following an acute injury to the central or peripheral nervous system,
wherein the adenosine and inosine are infused at an A:I ratio of about 1:1 to about 1:20 or at an A:I ratio of about 1:1 to about 20:1.

64. The method of claim 63, wherein the injury is an acute injury of the spinal cord.

65. The method of claim 63, wherein the injury is a stroke.

66. The method of claim 63, wherein adenosine and inosine are infused in a pharmaceutical composition comprising adenosine and inosine in an adenosine:inosine weight ratio of about 1:1 to about 1:20.

67. The method of claim 63 wherein adenosine and inosine are infused intravenously.

68. The method of claim 63, wherein adenosine is infused at a rate of less than about 80 μg/kg/min.

69. The method of claim 63, wherein adenosine is infused at a rate of 35-70 μg/kg/min.

70. The method of claim 63, wherein inosine is infused at 35-70 μg/kg/min.

71. The method of claim 63, wherein inosine is infused at 10-210 μg/kg/min.

72. The method of claim 63, wherein the first infusion is begun within 6 hours of onset of acute injury.

73. The method of claim 63, further comprising at least a second concurrent parenteral infusion of adenosine and inosine, wherein adenosine and inosine are infused at an A:I ratio of about 1:1 to about 1:20 or at an A:I ratio of about 1:1 to 20:1.

74. The method of claim 63, wherein each infusion is of at least 30 minutes duration.

75. A method of treating acute pulmonary vascular resistance, the method comprising:
administering at least a first concurrent parenteral infusion of adenosine and inosine during or following an acute cardiovascular or respiratory disorder, wherein the adenosine and inosine are infused at an A:I ratio of about 1:1 to about 1:20.

76. In a method of percutaneous transluminal coronary angioplasty or thrombolysis, the improvement comprising:
administering adenosine and inosine concurrently during the angioplasty or the thrombolysis, wherein adenosine and inosine are administered in an adenosine:inosine ratio of about 1:1 to about 1:20 or of about 1:1 to 20:1.

77. A method of increasing cardiac output, the method comprising:
administering at least a first concurrent parenteral infusion of adenosine and inosine at an adenosine:inosine ratio of 1:1 to 1:20, in an amount and for a duration sufficient to increase cardiac output.

78. A method of prophylaxis for post-operative complications, the method comprising:
administering adenosine and inosine concurrently intraoperatively or postoperatively during the intensive care unit period
wherein adenosine and inosine are administered in an adenosine:inosine weight ratio of about 1:1 to about 1:20 or in an adenosine:inosine weight ratio of about 1:1 to about 20:1.
